(19) United States
(12) Patent Application Publication
(10) Pub. No.: US 2023/0203596 A1
(43) Pub. Date: Jun. 29, 2023

(54) A METHOD OF DIAGNOSING, PROGNOSING AND/OR MONITORING OVARIAN CANCER

(71) Applicant: The Queen's University of Belfast, Belfast (GB)

(72) Inventors: Paul Mullan, Belfast (GB); James P. Beirne, Belfast (GB); Laura Feeney, Belfast (GB); W. Glenn McCluggage, Belfast (GB); Ian JG Harley, Belfast (GB)

(73) Assignee: The Queen's University of Belfast, Belfast (GB)

(21) Appl. No.: 18/000,178

(22) PCT Filed: May 28, 2021
(86) PCT No.: PCT/EP2021/064454

§ 371 Date: Nov. 29, 2022

(30) Foreign Application Priority Data
May 29, 2020 (GB) 2008113.9

(51) Int. Cl. C12Q 1/6886

(52) U.S. Cl. CPC C12Q 1/6886; C12Q 2600/118; C12Q 2600/154; C12Q 1/6851

(57) ABSTRACT

The present invention relates to a method of diagnosing ovarian cancer, including fallopian tube and/or primary peritoneal cancer. Specifically, the present invention relates to a method of diagnosing high grade serous carcinoma. The invention involves measuring the nucleic acid methylation levels of one or more biomarkers in the sample; and diagnosing ovarian cancer in the patient based on the nucleic acid methylation levels.

### FIELD OF THE INVENTION

The present invention relates to a method of diagnosing, prognosing and/or monitoring ovarian cancer, including fallopian tube and/or primary peritoneal cancer. Specifically, the present invention relates to a method of diagnosing, prognosing and/or monitoring high grade serous carcinoma.

### BACKGROUND TO THE INVENTION

Ovarian cancer (OC) is an umbrella term for various different types of cancer that affect the ovaries, fallopian tubes, and the peritoneal cavity. The majority of ovarian cancers (˜70%) are epithelial in nature. Epithelial ovarian cancer (EOC) encompasses five main types; high-grade serous carcinoma (HGSC) (70%), endometrioid carcinoma (EC) (10%), clear cell carcinoma (CCC) (10%), mucinous carcinomas (MC) (3%), and low-grade serous carcinomas (LGSC) (3%). EOC is the most common cause of death from gynaecological malignancy in the developed world, with most deaths being attributed to HGSC—the most common and most aggressive subtype of EOC. Although 5-year survival has improved over the past 30 years, the prognosis for EOC remains poor. It is typically associated with non-specific symptoms and, therefore, commonly diagnosed at an advanced stage, where there is a poor prognosis. Almost 75% of women with EOC present at a late stage (58% stage 3, 17% stage 4), with associated 5-year survival rates of approximately 35%. Approximately 25% of women present with early stage disease (20% stage 1, 5% stage 2). If diagnosed in the earliest stage (stage 1), survival is greatly improved—if detected at an early stage survival rates increase up to 80-95%. However, there is currently no effective method of screening for EOC.

Historically, most theories of the pathogenesis of OC included the concept that it begins with the dedifferentiation of the cells overlying the ovary; the ovarian surface epithelium (OSE). For decades the incessant ovulation theory was the most accepted hypothesis of OC carcinogenesis. However, there is a distinct lack of pathological evidence supporting this theory. One of the major advances in our understanding of the pathogenesis of OC was the recognition that a high proportion of HGSCs may originate from the epithelium of the distal fallopian tube, rather that the OSE. Beginning with the discovery of the BRCA-associated ovarian cancer susceptibility genes and subsequent examination of risk-reducing salpingo-oophorectomy (RRSO) specimens, a new model of ovarian carcinogenesis began to unfold drawing attention to the distal fallopian tube as a more likely site of origin for HGSC. HGSC is characterised by ubiquitous TP53 mutation. The most compelling evidence for the proposed new site of origin came from a series of confirmatory reports which identified early serous cancers containing TP53 mutations in the fallopian tube but not the ovary. Subsequent studies identified the presence of potential precursor lesions in the distal fallopian tube in high-risk women. Serous intraepithelial or early invasive carcinomas were found in up to 10% of fallopian tubes in BRCA mutation carriers who had undergone prophylactic bilateral salpingo-oophorectomies. These proliferations, termed serous tubal intraepithelial carcinomas (STIC), demonstrated identical TP53 mutations to adjacent HGSC. Following on from these initial studies, several studies have since reported the detection of STICs in up to 60% of women with HGSC, in both hereditary and sporadic cases. Thus, strong pathological evidence supports the theory that the distal fallopian tube is the origin of HGSC and the precursor lesion STIC has been identified.

Histopathological examination is considered the gold standard for OC diagnosis. However, current methods can be time consuming and costly. Also, accessing adequate tumour tissue can be challenging. The main diagnostic tools currently available are clinical (abdomino-pelvic examination), biochemical (serum tumour biomarkers such as cancer antigen 125 (CA125) or human epididymis protein 4 (HE4)), radiological (abdominopelvic and transvaginal ultrasound, cross-sectional imaging) and cytological (peritoneal or pleural fluid).

Since its discovery over thirty years ago, CA125 has remained the gold standard serum biomarker for OC. It is a glycoprotein antigen detected by using mouse monoclonal antibody 00125 raised from an OC cell line. CA125 is not specific to OC and is widely distributed in other adult tissues including the liver, kidneys, colon, pancreas and lungs. CA125 levels vary widely based on age and tend to be lower in postmenopausal women. There is also variation in levels depending on race, physiological factors and several benign and malignant conditions including: menstruation; endometriosis; pelvic inflammation; liver, renal, and lung disease; cancer of the endometrium, breast, colon, pancreas, lung, stomach, and liver. While CA125 levels are increased in about 80-85% of women with advanced ovarian cancer, only 50% of patients with early stage (stage 1) disease have elevated levels, thus limiting its utility as a screening test. Furthermore, CA125 is not expressed or produced in approximately 20% of OC. In the past decade, intensive efforts have been made to try to find more effective biomarkers for OC. Despite a multitude of potential biomarkers being investigated, substandard sensitivity and specificity has limited their translation into routine clinical practice.

Cancer screening has been shown to improve mortality rates in cancers such as breast, cervical, and colorectal cancer. Unlike the successful screening programmes that have been developed for these cancers, there is currently no acceptable programme for OC. This is in part due to the invasive nature of obtaining tissue samples from patients with suspected OC and, until recently, a lack of identifiable precancerous lesions. Furthermore, as OC has a relatively low prevalence rate, screening strategies require a high sensitivity (>75%) and specificity (99.6%) with a positive predictive value (PPV) of at least 10%. Multiple efforts have been made to improve survival rates through early screening methods based on serum CA125 levels and TVUS. Thus far, none of these methods have met the standards required to advocate population-based screening.

Precision oncology seeks to obtain molecular information about cancer to improve patient outcomes. Tissue biopsy samples are widely used to characterise tumours. However, this method, beyond initial diagnosis, of tumour analysis is limited by constraints on sampling frequency and incomplete representation of the entire tumour. In recent years, the focus of precision medicine is increasingly turning towards minimally invasive biopsies that can be repeated at multiple time points facilitating ‘real-time’ disease monitoring. The diagnosis of early stage cancer remains extremely challenging and this applies to ovarian cancer in particular, given its asymptomatic behaviour up until its usual advanced stages of clinical presentation. Recent research suggests that technological advances in the analysis of cell-free DNA (cfDNA) may provide a solution to these challenges. The main obstacles to the development of cfDNA-based biomarkers are: (1) low abundance of circulating tumour DNA (ctDNA) in the blood; and (2) high levels of background non-cancerous cfDNA, mostly shed from white blood cells (WBCs) in the blood. Highly sensitive technologies are required to accurately detect scarcely abundant alleles within high background levels of non-target molecules.

Identifying and developing novel biomarkers for minimally-invasive detection of OC and prognosis of HGSC would fulfil an unmet clinical need in a poor outcome cancer. The use of such biomarkers in the tissue pathology arena will not only improve diagnostic accuracy but also quicken the diagnostic process and allow real time monitoring of ovarian cancer tumour burden throughout the treatment process and potential relapses in routine follow up clinical visits.

### SUMMARY OF THE INVENTION

According to a first aspect of the present invention, there is provided a method of diagnosing ovarian cancer in a patient, the method comprising the steps of:
(a) providing a biological sample from the patient;
(b) measuring the nucleic acid methylation levels of one or more biomarkers in the sample; and
(c) diagnosing ovarian cancer in the patient based on the nucleic acid methylation levels.

According to a second aspect of the present invention, there is provided a method of prognosing ovarian cancer in a patient, the method comprising the steps of:
(a) providing a biological sample from the patient;
(b) measuring the nucleic acid methylation levels of one or more biomarkers in the sample; and
(c) prognosing ovarian cancer in the patient based on the nucleic acid methylation levels.

Optionally, the ovarian cancer is fallopian tube cancer.

Optionally or additionally, the ovarian cancer is primary peritoneal cancer.

Optionally, the ovarian cancer is epithelial ovarian cancer.

Optionally, the ovarian cancer is selected from high grade serous carcinoma, low grade serous carcinoma, clear cell carcinoma, endometrioid carcinoma, and mucinous carcinoma.

Preferably, the ovarian cancer is high grade serous carcinoma.

Optionally, the ovarian cancer is serous tubal intraepithelial carcinoma (STIC), or serous tubal intraepithelial lesion (STIL).

Optionally, the or each biomarker is a nucleic acid. Further optionally, the or each biomarker is a deoxyribonucleic acid. Still further optionally, the or each biomarker is a ribonucleic acid.

Preferably, the or each biomarker is a deoxyribonucleic acid.

Optionally, the or each biomarker is a gene.

Optionally, the or each biomarker is a gene selected from KRT87P; OSR2; LINC01197; ZNF469; MAP3K8; LINC01798; PHACTR3; TNS3; TFAP2A/LINC00518; PRRX1; NR5A1; LHX9; RBPMS; TACC1; DNHD1; TGFB1 I1; CACNA1A; ZNF154; KRT87P; PHOX2B; SHANK1; GRM1; HOXA9; TCERG1L; SCGB1B2P/LOC643719; NEFM; BARHL2; ZIC1; CLIC6; HCG15; PCDHGA4; LINC01159; PCDHGA4; CLIC6; ZNF729; PANTR1/LINC01158; DLX1/DLX-DT; and OTX1.

Optionally, the or each biomarker is a gene selected from KRT87P; OSR2; LINC01197; ZNF469; MAP3K8; LINC01798; PHACTR3; TNS3; TFAP2A/LINC00518; PRRX1; NR5A1; LHX9; RBPMS; TACC1; DNHD1; TGFB1 I1; CACNA1A; ZNF154 and KRT87P.

Optionally, the or each biomarker is a gene selected from KRT87P; OSR2; TFAP2A/LINC00518; NR5A1; PHACTR3; PRRX1; MAP3K8; LINC01798; ZNF154; and TGFB111.

Optionally, the or each biomarker is a gene selected from KRT87P; OSR2; TFAP2A/LINC00518; NR5A1; PRRX1; LINC01798; ZNF154; and TGFB111.

Optionally, the or each biomarker is a gene selected from KRT87P; OSR2; TFAP2A/LINC00518; PRRX1; ZNF154; and TGFB111.

Optionally, the or each biomarker is a gene selected from KRT87P; OSR2; PRRX1; ZNF154; and LINC01798.

Optionally, the or each biomarker is a gene selected from KRT87P; OSR2; ZNF154; and PRRX1.

Preferably, the biomarker is the KRT87P gene.

Optionally or additionally, the or each biomarker is a gene selected from TFAP2A/LINC00518; PHOX2B; SHANK1; GRM1; HOXA9; TCERG1L; SCGB1B2P/LOC643719; NEFM; BARHL2; ZIC1; CLIC6; HCG15; PCDHGA4; LINC01159; PCDHGA4; CLIC6; ZNF729; PANTR1/LINC01158; DLX1/DLX-DT; and OTX1.

Optionally, the or each biomarker is a gene selected from KRT87P; OSR2; TFAP2A/LINC00518; NR5A1; PHACTR3; PRRX1; MAP3K8; LINC01798; ZNF154; TGFB1 I1; PHOX2B; SHANK1; GRM1; HOXA9; TCERG1L; SCGB1B2P/LOC643719; NEFM; BARHL2; ZIC1; CLIC6; HCG15; PCDHGA4; LINC01159; PCDHGA4; CLIC6; ZNF729; PANTR1/LINC01158; DLX1/DLX-DT; and OTX1.

Optionally, the or each biomarker is a gene selected from KRT87P; OSR2; TFAP2A/LINC00518; NR5A1; PRRX1; LINC01798; ZNF154; TGFB1I1; PHOX2B; SHANK1; GRM1; HOXA9; TCERG1L; SCGB1B2P/LOC643719; NEFM; BARHL2; ZIC1; CLIC6; HCG15; PCDHGA4; LINC01159; PCDHGA4; CLIC6; ZNF729; PANTR1/LINC01158; DLX1/DLX-DT; and OTX1.

Optionally, the or each biomarker is a gene selected from KRT87P; OSR2; TFAP2A/LINC00518; PRRX1; ZNF154; TGFB1I1; PHOX2B; SHANK1; GRM1; HOXA9; TCERG1L; SCGB1B2P/LOC643719; NEFM; BARHL2; ZIC1; CLIC6; HCG15; PCDHGA4; LINC01159; PCDHGA4; CLIC6; ZNF729; PANTR1/LINC01158; DLX1/DLX-DT; and OTX1.

Optionally, the or each biomarker is a gene selected from KRT87P; OSR2; PRRX1; ZNF154; LINC01798; PHOX2B; SHANK1; GRM1; HOXA9; TCERG1L; SCGB1B2P/LOC643719; NEFM; BARHL2; ZIC1; CLIC6; HCG15; PCDHGA4; LINC01159; PCDHGA4; CLIC6; ZNF729; PANTR1/LINC01158; DLX1/DLX-DT; and OTX1.

Optionally, the or each biomarker is a gene selected from KRT87P; OSR2; ZNF154; PRRX1; PHOX2B; SHANK1; GRM1; HOXA9; TCERG1L; SCGB1B2P/LOC643719; NEFM; BARHL2; ZIC1; CLIC6; HCG15; PCDHGA4; LINC01159; PCDHGA4; CLIC6; ZNF729; PANTR1/LINC01158; DLX1/DLX-DT; and OTX1.

Optionally or additionally, the or each biomarker is a gene selected from PHOX2B; SHANK1; GRM1; HOXA9; TCERG1L; SCGB1B2P/LOC643719; NEFM; BARHL2; ZIC1; and CLIC6.

Optionally, the or each biomarker is a gene selected from KRT87P; OSR2; TFAP2A/LINC00518; NR5A1; PHACTR3; PRRX1; MAP3K8; LINC01798; ZNF154; TGFB1 I1; PHOX2B; SHANK1; GRM1; HOXA9; TCERG1L; SCGB1B2P/LOC643719; NEFM; BARHL2; ZIC1; and CLIC6.

Optionally, the or each biomarker is a gene selected from KRT87P; OSR2; TFAP2A/LINC00518; NR5A1; PRRX1; LINC01798; ZNF154; TGFB1I1; PHOX2B; SHANK1; GRM1; HOXA9; TCERG1L; SCGB1B2P/LOC643719; NEFM; BARHL2; ZIC1; and CLIC6.

Optionally, the or each biomarker is a gene selected from KRT87P; OSR2; TFAP2A/LINC00518; PRRX1; ZNF154; TGFB1 I1; PHOX2B; SHANK1; GRM1; HOXA9; TCERG1L; SCGB1B2P/LOC643719; NEFM; BARHL2; ZIC1; and CLIC6.

Optionally, the or each biomarker is a gene selected from KRT87P; OSR2; PRRX1; ZNF154; LINC01798; PHOX2B; SHANK1; GRM1; HOXA9; TCERG1L; SCGB1B2P/LOC643719; NEFM; BARHL2; ZIC1; CLIC6; HCG15; PCDHGA4; LINC01159; PCDHGA4; and CLIC6.

Optionally, the or each biomarker is a gene selected from KRT87P; OSR2; ZNF154; PRRX1; PHOX2B; SHANK1; GRM1; HOXA9; TCERG1L; SCGB1B2P/LOC643719; NEFM; BARHL2; ZIC1; and CLIC6.

Optionally, the or each biomarker is a gene having a NCBI Reference Sequence Version Number selected from NR_146088.1; NM_001286841.1; NR_034095.1; NM_001367624.2; NM_001320961.2; NR_110156.1; NM_080672.5; NM_022748.12; NM_001372066.1, NR_027793.1; NM_006902.5; NM_004959.5; NM_020204.3; NM_001008710.3; NM_001352789.2; NM_144666.3; NM_001042454.3; NM_001127222.2; NM_001085384.3 and NM_001320198.2; NM_003924; NM_016148; NM_001278064; NM_152739; NM_174937; NR_027620; NM_005382; NM_020063; NM_003412; NM_001317009; NR_145490; NM_018917; NR_110373; NM_018917; NM_001317009; NM_001242680; NR_037883; NM_178120; and NM_014562.

Optionally, the or each biomarker is a gene having a NCBI Reference Sequence Version Number selected from NR_146088.1; NM_001286841.1; NR_034095.1; NM_001367624.2; NM_001320961.2; NR_110156.1; NM_080672.5; NM_022748.12; NM_001372066.1, NR_027793.1; NM_006902.5; NM_004959.5; NM_020204.3; NM_001008710.3; NM_001352789.2; NM_144666.3; NM_001042454.3; NM_001127222.2; NM_001085384.3 and NM_001320198.2.

Optionally, the or each biomarker is a gene having a NCBI Reference Sequence Version Number selected from NR_146088.1; NM_001286841.1; NM_001372066.1, NR_027793.1, NM_004959.5; NM_080672.5; NM_006902.5; NM_001320961.2; NR_110156.1; NM_001085384.3; and NM_001042454.3.

Optionally, the or each biomarker is a gene having a NCBI Reference Sequence Version Number selected from NR_146088.1; NM_001286841.1; NM_001372066.1, NR_027793.1, NM_004959.5; NM_006902.5; NR_110156.1; NM_001085384.3; and NM_001042454.3.

Optionally, the or each biomarker is a gene having a NCBI Reference Sequence Version Number selected from NR_146088.1; NM_001286841.1; NM_001372066.1, NR_027793.1, NM_006902.5; NM_001085384.3; and NM_001042454.3.

Optionally, the or each biomarker is a gene having a NCBI Reference Sequence Version Number selected from NR_146088.1; NM_001286841.1; NM_006902.5; NM_001085384.3; and NR_110156.1.

Optionally, the or each biomarker is a gene having a NCBI Reference Sequence Version Number selected from NR_146088.1; NM_001286841.1; NM_001085384.3; and NM_006902.5.

Preferably, the biomarker is the gene having the NCBI Reference Sequence Version Number NR_146088.1.

Optionally or additionally, the or each biomarker is a gene having a NCBI Reference Sequence Version Number selected from NM_001372066.1; NM_003924; NM_016148; NM_001278064; NM_152739; NM_174937; NR_027620; NM_005382; NM_020063; NM_003412; NM_001317009; NR_145490; NM_018917; NR_110373; NM_018917; NM_001317009; NM_001242680; NR_037883; NM_178120; and NM_014562.

Optionally, the or each biomarker is a gene having a NCBI Reference Sequence Version Number selected from NR_146088.1; NM_001286841.1; NR_034095.1; NM_001367624.2; NM_001320961.2; NR_110156.1; NM_080672.5; NM_022748.12; NM_001372066.1, NR_027793.1; NM_006902.5; NM_004959.5; NM_020204.3; NM_001008710.3; NM_001352789.2; NM_144666.3; NM_001042454.3; NM_001127222.2; NM_001085384.3; NM_001320198.2; and NM_003924; NM_016148; NM_001278064; NM_152739; NM_174937; NR_027620; NM_005382; NM_020063; NM_003412; NM_001317009; NR_145490; NM_018917; NR_110373; NM_018917; NM_001317009; NM_001242680; NR_037883; NM_178120; and NM_014562.

Optionally, the or each biomarker is a gene having a NCBI Reference Sequence Version Number selected from NR_146088.1; NM_001286841.1; NM_001372066.1, NR_027793.1, NM_004959.5; NM_080672.5; NM_006902.5; NM_001320961.2; NR_110156.1; NM_001085384.3; NM_001042454.3; and NM_003924; NM_016148; NM_001278064; NM_152739; NM_174937; NR_027620; NM_005382; NM_020063; NM_003412; NM_001317009; NR_145490; NM_018917; NR_110373; NM_018917; NM_001317009; NM_001242680; NR_037883; NM_178120; and NM_014562.

Optionally, the or each biomarker is a gene having a NCBI Reference Sequence Version Number selected from NR_146088.1; NM_001286841.1; NM_001372066.1, NR_027793.1, NM_004959.5; NM_006902.5; NR_110156.1; NM_001085384.3; NM_001042454.3; and NM_003924; NM_016148; NM_001278064; NM_152739; NM_174937; NR_027620; NM_005382; NM_020063; NM_003412; NM_001317009; NR_145490; NM_018917; NR_110373; NM_018917; NM_001317009; NM_001242680; NR_037883; NM_178120; and NM_014562.

Optionally, the or each biomarker is a gene having a NCBI Reference Sequence Version Number selected from NR_146088.1; NM_001286841.1; NM_001372066.1, NR_027793.1, NM_006902.5; NM_001085384.3; NM_001042454.3; NM_003924; NM_016148; NM_001278064; NM_152739; NM_174937; NR_027620; NM_005382; NM_020063; NM_003412; NM_001317009; NR_145490; NM_018917; NR_110373; NM_018917; NM_001317009; NM_001242680; NR_037883; NM_178120; and NM_014562.

Optionally, the or each biomarker is a gene having a NCBI Reference Sequence Version Number selected from NR_146088.1; NM_001286841.1; NM_006902.5; NM_001085384.3; NR_110156.1; NM_003924; NM_016148; NM_001278064; NM_152739; NM_174937; NR_027620; NM_005382; NM_020063; NM_003412; NM_001317009; NR_145490; NM_018917; NR_110373; NM_018917; NM_001317009; NM_001242680; NR_037883; NM_178120; and NM_014562.

Optionally, the or each biomarker is a gene having a NCBI Reference Sequence Version Number selected from NR_146088.1; NM_001286841.1; NM_001085384.3; NM_006902.5; and NM_003924; NM_016148; NM_001278064; NM_152739; NM_174937; NR_027620; NM_005382; NM_020063; NM_003412; NM_001317009; NR_145490; NM_018917; NR_110373; NM_018917; NM_001317009; NM_001242680; NR_037883; NM_178120; and NM_014562.

Optionally, the or each biomarker is a gene having a NCBI Reference Sequence Version Number selected from NR_146088.1; NM_001286841.1; NR_034095.1; NM_001367624.2; NM_001320961.2; NR_110156.1; NM_080672.5; NM_022748.12; NM_001372066.1, NR_027793.1; NM_006902.5; NM_004959.5; NM_020204.3; NM_001008710.3; NM_001352789.2; NM_144666.3; NM_001042454.3; NM_001127222.2; NM_001085384.3; NM_001320198.2; and NM_003924; NM_016148; NM_001278064; NM_152739; NM_174937; NR_027620; NM_005382; NM_020063; NM_003412; and NM_001317009.

Optionally, the or each biomarker is a gene having a NCBI Reference Sequence Version Number selected from NR_146088.1; NM_001286841.1; NM_001372066.1, NR_027793.1, NM_004959.5; NM_080672.5; NM_006902.5; NM_001320961.2; NR_110156.1; NM_001085384.3; NM_001042454.3; and NM_003924; NM_016148; NM_001278064; NM_152739; NM_174937; NR_027620; NM_005382; NM_020063; NM_003412; and NM_001317009.

Optionally, the or each biomarker is a gene having a NCBI Reference Sequence Version Number selected from NR_146088.1; NM_001286841.1; NM_001372066.1, NR_027793.1, NM_004959.5; NM_006902.5; NR_110156.1; NM_001085384.3; NM_001042454.3; and NM_003924; NM_016148; NM_001278064; NM_152739; NM_174937; NR_027620; NM_005382; NM_020063; NM_003412; and NM_001317009.

Optionally, the or each biomarker is a gene having a NCBI Reference Sequence Version Number selected from NR_146088.1; NM_001286841.1; NM_001372066.1, NR_027793.1, NM_006902.5; NM_001085384.3; NM_001042454.3; NM_003924; NM_016148; NM_001278064; NM_152739; NM_174937; NR_027620; NM_005382; NM_020063; NM_003412; NM_001317009; NR_145490; NM_018917; NR_110373; NM_018917; and NM_001317009.

Optionally, the or each biomarker is a gene having a NCBI Reference Sequence Version Number selected from NR_146088.1; NM_001286841.1; NM_006902.5; NM_001085384.3; NR_110156.1; NM_003924; NM_016148; NM_001278064; NM_152739; NM_174937; NR_027620; NM_005382; NM_020063; NM_003412; and NM_001317009.

Optionally, the or each biomarker is a gene having a NCBI Reference Sequence Version Number selected from NR_146088.1; NM_001286841.1; NM_001085384.3; NM_006902.5; and NM_003924; NM_016148; NM_001278064; NM_152739; NM_174937; NR_027620; NM_005382; NM_020063; NM_003412; and NM_001317009.

Optionally, the measuring step (b) comprises measuring a methyl group of the or each biomarker. Further optionally, the measuring step (b) comprises measuring a methyl group of the or each deoxyribonucleic acid. Still further optionally, the measuring step (b) comprises measuring a methyl group of the or each gene.

Optionally, the measuring step (b) comprises measuring a methyl group of a cytosine/guanine dinucleotide of the or each biomarker. Further optionally, the measuring step (b) comprises measuring a methyl group of a methyl group of a cytosine/guanine dinucleotide of the or each deoxyribonucleic acid. Still further optionally, the measuring step (b) comprises measuring a methyl group of a methyl group of a cytosine/guanine dinucleotide of the or each gene.

Optionally, the measuring step (b) comprises measuring a methyl group of one or more cytosine/guanine dinucleotide having a CpG cluster ID (cg #) selected from cg07078225; cg08202494; cg01657761; cg03035213; cg03314029; cg04453471; cg07215504; cg11469908; cg15712559; cg16329896; cg05224741; cg09010107; cg04043571; cg08610862; cg13912311; cg23044884; cg14284618; cg15511120; cg23910243; cg22187630; cg01268824; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally, the measuring step (b) comprises measuring a methyl group of one or more cytosine/guanine dinucleotide having a CpG cluster ID (cg #) selected from cg07078225; cg08202494; cg01657761; cg03035213; cg03314029; cg04453471; cg07215504; cg11469908; cg15712559; cg16329896; cg05224741; cg09010107; cg04043571; cg08610862; cg13912311; cg23044884; cg14284618; cg15511120; cg23910243; cg22187630; and cg01268824.

Optionally, the measuring step (b) comprises measuring a methyl group of one or more cytosine/guanine dinucleotide having a CpG cluster ID (cg #) selected from cg08202494; cg05224741; cg13912311; cg15712559; cg09010107; cg04453471; cg11469908; cg01268824; cg07078225 and cg23910243.

Optionally, the measuring step (b) comprises measuring a methyl group of one or more cytosine/guanine dinucleotide having a CpG cluster ID (cg #) selected from cg08202494; cg05224741; cg13912311; cg05224741; cg09010107; cg11469908; cg01268824; cg07078225 and cg23910243.

Optionally, the measuring step (b) comprises measuring a methyl group of one or more cytosine/guanine dinucleotide having a CpG cluster ID (cg #) selected from cg08202494; cg05224741; cg09010107; cg05224741; cg07078225 and cg23910243.

Optionally, the measuring step (b) comprises measuring a methyl group of one or more cytosine/guanine dinucleotide having a CpG cluster ID (cg #) selected from cg08202494; cg09010107; cg05224741; and cg07078225.

Optionally, the measuring step (b) comprises measuring a methyl group of one or more cytosine/guanine dinucleotide having a CpG cluster ID (cg #) selected from cg08202494; cg07078225 and cg09010107.

Optionally, the measuring step (b) comprises measuring a methyl group of one or more cytosine/guanine dinucleotide having a CpG cluster ID (cg #) selected from cg07078225 and cg09010107.

Optionally, the measuring step (b) comprises measuring a methyl group of the cytosine/guanine dinucleotide having a CpG cluster ID (cg #) cg07078225.

Preferably, the measuring step (b) comprises measuring a methyl group of one or more cytosine/guanine dinucleotide having a CpG cluster ID (cg #) selected from cg07078225, cg05224741 and cg08202494.

Further preferably, the measuring step (b) comprises measuring a methyl group of one or more cytosine/guanine dinucleotide having a CpG cluster ID (cg #) selected from cg07078225 and cg05224741.

Most preferably, the measuring step (b) comprises measuring a methyl group of one or more cytosine/guanine dinucleotide having a CpG cluster ID (cg #) cg07078225.

Optionally or additionally, the measuring step (b) comprises measuring a methyl group of one or more cytosine/guanine dinucleotide having a CpG cluster ID (cg #) selected from cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally, the measuring step (b) comprises measuring a methyl group of one or more cytosine/guanine dinucleotide having a CpG cluster ID (cg #) selected from cg08202494; cg01657761; cg03035213; cg03314029; cg04453471; cg07215504; cg11469908; cg15712559; cg16329896; cg05224741; cg09010107; cg04043571; cg08610862; cg13912311; cg23044884; cg14284618; cg15511120; cg23910243; cg22187630; cg01268824; cg07078225; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally, the measuring step (b) comprises measuring a methyl group of one or more cytosine/guanine dinucleotide having a CpG cluster ID (cg #) selected from cg08202494; cg05224741; cg13912311; cg15712559; cg09010107; cg04453471; cg11469908; cg01268824; cg07078225; cg23910243; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally, the measuring step (b) comprises measuring a methyl group of one or more cytosine/guanine dinucleotide having a CpG cluster ID (cg #) selected from cg08202494; cg05224741; cg13912311; cg05224741; cg09010107; cg11469908; cg01268824; cg07078225; cg23910243; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally, the measuring step (b) comprises measuring a methyl group of one or more cytosine/guanine dinucleotide having a CpG cluster ID (cg #) selected from cg08202494; cg05224741; cg09010107; cg05224741; cg07078225; cg23910243; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally, the measuring step (b) comprises measuring a methyl group of one or more cytosine/guanine dinucleotide having a CpG cluster ID (cg #) selected from cg08202494; cg09010107; cg05224741; cg07078225; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally, the measuring step (b) comprises measuring a methyl group of one or more cytosine/guanine dinucleotide having a CpG cluster ID (cg #) selected from cg08202494; cg07078225; cg09010107; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally or additionally, the measuring step (b) comprises measuring a methyl group of one or more cytosine/guanine dinucleotide having a CpG cluster ID (cg #) selected from cg07078225; cg07907386; cg08447324; cg08958294; cg26365299; cg03109827; cg23200020; cg23290344; cg24884703; cg12595013; and cg11528328.

Optionally, the measuring step (b) comprises measuring a methyl group of one or more cytosine/guanine dinucleotide having a CpG cluster ID (cg #) selected from cg08202494; cg01657761; cg03035213; cg03314029; cg04453471; cg07215504; cg11469908; cg15712559; cg16329896; cg05224741; cg09010107; cg04043571; cg08610862; cg13912311; cg23044884; cg14284618; cg15511120; cg23910243; cg22187630; cg01268824; cg07078225; cg07907386; cg08447324; cg08958294; cg26365299; cg03109827; cg23200020; cg23290344; cg24884703; cg12595013; and cg11528328.

Optionally, the measuring step (b) comprises measuring a methyl group of one or more cytosine/guanine dinucleotide having a CpG cluster ID (cg #) selected from cg08202494; cg05224741; cg13912311; cg15712559; cg09010107; cg04453471; cg11469908; cg01268824; cg07078225; cg23910243; cg07907386; cg08447324; cg08958294; cg26365299; cg03109827; cg23200020; cg23290344; cg24884703; cg12595013; and cg11528328.

Optionally, the measuring step (b) comprises measuring a methyl group of one or more cytosine/guanine dinucleotide having a CpG cluster ID (cg #) selected from cg08202494; cg05224741; cg13912311; cg05224741; cg09010107; cg11469908; cg01268824; cg07078225; cg23910243; cg07907386; cg08447324; cg08958294; cg26365299; cg03109827; cg23200020; cg23290344; cg24884703; cg12595013; and cg11528328.

Optionally, the measuring step (b) comprises measuring a methyl group of one or more cytosine/guanine dinucleotide having a CpG cluster ID (cg #) selected from cg08202494; cg05224741; cg09010107; cg05224741; cg07078225; cg23910243; cg07907386; cg08447324; cg08958294; cg26365299; cg03109827; cg23200020; cg23290344; cg24884703; cg12595013; and cg11528328.

Optionally, the measuring step (b) comprises measuring a methyl group of one or more cytosine/guanine dinucleotide having a CpG cluster ID (cg #) selected from cg08202494; cg09010107; cg05224741; cg07078225; cg07907386; cg08447324; cg08958294; cg26365299; cg03109827; cg23200020; cg23290344; cg24884703; cg12595013; and cg11528328.

Optionally, the measuring step (b) comprises measuring a methyl group of one or more cytosine/guanine dinucleotide having a CpG cluster ID (cg #) selected from cg08202494; cg07078225; cg09010107; cg07907386; cg08447324; cg08958294; cg26365299; cg03109827; cg23200020; cg23290344; cg24884703; cg12595013; and cg11528328.

Optionally, the measuring step (b) comprises measuring a methyl group of a cytosine nucleotide of a cytosine/guanine dinucleotide of the or each biomarker. Further optionally, the measuring step (b) comprises measuring a methyl group of a cytosine nucleotide of a cytosine/guanine dinucleotide of the or each deoxyribonucleic acid. Still further optionally, the measuring step (b) comprises measuring a methyl group of a cytosine nucleotide of a cytosine/guanine dinucleotide of the or each gene.

Optionally, the measuring step (b) comprises measuring a methyl group of a cytosine nucleotide of one or more cytosine/guanine dinucleotide having CpG cluster ID (cg #) selected from cg08202494; cg01657761; cg03035213; cg03314029; cg04453471; cg07215504; cg11469908; cg15712559; cg16329896; cg05224741; cg09010107; cg04043571; cg08610862; cg13912311; cg23044884; cg14284618; cg15511120; cg23910243; cg22187630; cg01268824; cg07078225; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally, the measuring step (b) comprises measuring a methyl group of a cytosine nucleotide of one or more cytosine/guanine dinucleotide having CpG cluster ID (cg #) selected from cg08202494; cg01657761; cg03035213; cg03314029; cg04453471; cg07215504; cg11469908; cg15712559; cg16329896; cg05224741; cg09010107; cg04043571; cg08610862; cg13912311; cg23044884; cg14284618; cg15511120; cg23910243; cg22187630; cg01268824 and cg07078225.

Optionally, the measuring step (b) comprises measuring a methyl group a cytosine nucleotide of one or more cytosine/guanine dinucleotide having a CpG cluster ID (cg #) selected from cg08202494; cg05224741; cg13912311; cg15712559; cg09010107; cg04453471; cg11469908; cg01268824; cg07078225 and cg23910243.

Optionally, the measuring step (b) comprises measuring a methyl group a cytosine nucleotide of one or more cytosine/guanine dinucleotide having a CpG cluster ID (cg #) selected from cg08202494; cg05224741; cg13912311; cg09010107; cg11469908; cg01268824; cg07078225 and cg23910243.

Optionally, the measuring step (b) comprises measuring a methyl group a cytosine nucleotide of one or more cytosine/guanine dinucleotide having a CpG cluster ID (cg #) selected from cg08202494; cg05224741; cg09010107; cg01268824; cg07078225 and cg23910243.

Optionally, the measuring step (b) comprises measuring a methyl group a cytosine nucleotide of one or more cytosine/guanine dinucleotide having a CpG cluster ID (cg #) selected from cg08202494; cg09010107; cg05224741; cg07078225 and cg11469908.

Optionally, the measuring step (b) comprises measuring a methyl group a cytosine nucleotide of one or more cytosine/guanine dinucleotide having a CpG cluster ID (cg #) selected from cg08202494; cg07078225 and cg05224741.

Optionally, the measuring step (b) comprises measuring a methyl group a cytosine nucleotide of one or more cytosine/guanine dinucleotide having a CpG cluster ID (cg #) selected from cg05224741; and cg07078225.

Preferably, the measuring step (b) comprises measuring a methyl group a cytosine nucleotide of the cytosine/guanine dinucleotide having a CpG cluster ID (cg #) cg07078225.

Optionally or additionally, the measuring step (b) comprises measuring a methyl group a cytosine nucleotide of one or more cytosine/guanine dinucleotide having a CpG cluster ID (cg #) selected from cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally, the measuring step (b) comprises measuring a methyl group a cytosine nucleotide of one or more cytosine/guanine dinucleotide having a CpG cluster ID (cg #) selected from cg08202494; cg01657761; cg03035213; cg03314029; cg04453471; cg07215504; cg11469908; cg15712559; cg16329896; cg05224741; cg09010107; cg04043571; cg08610862; cg13912311; cg23044884; cg14284618; cg15511120; cg23910243; cg22187630; cg01268824; cg07078225; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally, the measuring step (b) comprises measuring a methyl group a cytosine nucleotide of one or more cytosine/guanine dinucleotide having a CpG cluster ID (cg #) selected from cg08202494; cg05224741; cg13912311; cg15712559; cg09010107; cg04453471; cg11469908; cg01268824; cg07078225; cg23910243; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally, the measuring step (b) comprises measuring a methyl group a cytosine nucleotide of one or more cytosine/guanine dinucleotide having a CpG cluster ID (cg #) selected from cg08202494; cg05224741; cg13912311; cg05224741; cg09010107; cg11469908; cg01268824; cg07078225; cg23910243; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally, the measuring step (b) comprises measuring a methyl group a cytosine nucleotide of one or more cytosine/guanine dinucleotide having a CpG cluster ID (cg #) selected from cg08202494; cg05224741; cg09010107; cg05224741; cg07078225; cg23910243; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally, the measuring step (b) comprises measuring a methyl group a cytosine nucleotide of one or more cytosine/guanine dinucleotide having a CpG cluster ID (cg #) selected from cg08202494; cg09010107; cg05224741; cg07078225; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally, the measuring step (b) comprises measuring a methyl group a cytosine nucleotide of one or more cytosine/guanine dinucleotide having a CpG cluster ID (cg #) selected from cg08202494; cg07078225; cg09010107; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

Optionally or additionally, the measuring step (b) comprises measuring a methyl group a cytosine nucleotide of one or more cytosine/guanine dinucleotide having a CpG cluster ID (cg #) selected from cg07907386; cg08447324; cg08958294; cg26365299; cg03109827; cg23200020; cg23290344; cg24884703; cg12595013; and cg11528328.

Optionally, the measuring step (b) comprises measuring a methyl group a cytosine nucleotide of one or more cytosine/guanine dinucleotide having a CpG cluster ID (cg #) selected from cg08202494; cg01657761; cg03035213; cg03314029; cg04453471; cg07215504; cg11469908; cg15712559; cg16329896; cg05224741; cg09010107; cg04043571; cg08610862; cg13912311; cg23044884; cg14284618; cg15511120; cg23910243; cg22187630; cg01268824; cg07078225; cg07907386; cg08447324; cg08958294; cg26365299; cg03109827; cg23200020; cg23290344; cg24884703; cg12595013; and cg11528328.

Optionally, the measuring step (b) comprises measuring a methyl group a cytosine nucleotide of one or more cytosine/guanine dinucleotide having a CpG cluster ID (cg #) selected from cg08202494; cg05224741; cg13912311; cg15712559; cg09010107; cg04453471; cg11469908; cg01268824; cg07078225; cg23910243; cg07907386; cg08447324; cg08958294; cg26365299; cg03109827; cg23200020; cg23290344; cg24884703; cg12595013; and cg11528328.

Optionally, the measuring step (b) comprises measuring a methyl group a cytosine nucleotide of one or more cytosine/guanine dinucleotide having a CpG cluster ID (cg #) selected from cg08202494; cg05224741; cg13912311; cg05224741; cg09010107; cg11469908; cg01268824; cg07078225; cg23910243; cg07907386; cg08447324; cg08958294; cg26365299; cg03109827; cg23200020; cg23290344; cg24884703; cg12595013; and cg11528328.

Optionally, the measuring step (b) comprises measuring a methyl group a cytosine nucleotide of one or more cytosine/guanine dinucleotide having a CpG cluster ID (cg #) selected from cg08202494; cg05224741; cg09010107; cg05224741; cg07078225; cg23910243; cg07907386; cg08447324; cg08958294; cg26365299; cg03109827; cg23200020; cg23290344; cg24884703; cg12595013; and cg11528328.

Optionally, the measuring step (b) comprises measuring a methyl group a cytosine nucleotide of one or more cytosine/guanine dinucleotide having a CpG cluster ID (cg #) selected from cg08202494; cg09010107; cg05224741; cg07078225; cg07907386; cg08447324; cg08958294; cg26365299; cg03109827; cg23200020; cg23290344; cg24884703; cg12595013; and cg11528328.

Optionally, the measuring step (b) comprises measuring a methyl group a cytosine nucleotide of one or more cytosine/guanine dinucleotide having a CpG cluster ID (cg #) selected from cg08202494; cg07078225; cg09010107; cg05224741; cg07907386; cg08447324; cg08958294; cg26365299; cg03109827; cg23200020; cg23290344; cg24884703; cg12595013; and cg11528328.

Optionally, the measuring step (b) comprises measuring a methyl group of a cytosine nucleotide of the or each biomarker. Further optionally, the measuring step (b) comprises measuring a methyl group of a cytosine nucleotide of the or each deoxyribonucleic acid. Still further optionally, the measuring step (b) comprises measuring a methyl group of a cytosine nucleotide of the or each gene.

Optionally, the measuring step (b) comprises measuring a methyl group of a nucleotide located at one or more of: chr8:99,961,381-99,961,763 (encompassing cg08202494); chr15:95,836,182-95,836,449 (encompassing cg01657761); chr16:88,496,985-88,497,220 (encompassing cg03035213); chr10:30,726,381-30,726,735 (encompassing cg04453471); chr16:88,496,945-88,497,180 (encompassing cg07215504); chr2:66,918,184-66,918,501 (encompassing cg11469908); chr20:58,180,486-58,180,815 (encompassing cg15712559); chr7:47,515,002-47,515,209 (encompassing cg16329896); chr6:10,422,232-10,422,455 (encompassing cg05224741); chr1:170,638,675-170,639,000 (encompassing cg09010107); chr9:127,265,620-127,266,359 (encompassing cg04043571); chr1:197,888,169-197,888,821 (encompassing cg08610862); chr9:127,265,221-127,265,590 (encompassing cg13912311); chr8:30,244,794-30,245,560 (encompassing cg23044884); chr8:38,627,724-38,628,106 (encompassing cg14284618); chr11:6,597,801-6,598,507 (encompassing cg15511120); chr16:31,484,429-31,484,901 (encompassing cg23910243); chr19:13,616,758-13,617,066 (encompassing cg22187630); chr19:58,220,672-58,220,980 (encompassing cg01268824); chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a nucleotide located at one or more of: chr8:99,961,381-99,961,763 (encompassing cg08202494); chr15:95,836,182-95,836,449 (encompassing cg01657761); chr16:88,496,985-88,497,220 (encompassing cg03035213); chr10:30,726,381-30,726,735 (encompassing cg04453471); chr16:88,496,945-88,497,180 (encompassing cg07215504); chr2:66,918,184-66,918,501 (encompassing cg11469908); chr20:58,180,486-58,180,815 (encompassing cg15712559); chr7:47,515,002-47,515,209 (encompassing cg16329896); chr6:10,422,232-10,422,455 (encompassing cg05224741); chr1:170,638,675-170,639,000 (encompassing cg09010107); chr9:127,265,620-127,266,359 (encompassing cg04043571); chr1:197,888,169-197,888,821 (encompassing cg08610862); chr9:127,265,221-127,265,590 (encompassing cg13912311); chr8:30,244,794-30,245,560 (encompassing cg23044884); chr8:38,627,724-38,628,106 (encompassing cg14284618); chr11:6,597,801-6,598,507 (encompassing cg15511120); chr16:31,484,429-31,484,901 (encompassing cg23910243); chr19:13,616,758-13,617,066 (encompassing cg22187630); chr19:58,220,672-58,220,980 (encompassing cg01268824); chr12:52,652,239-52,652,588 (encompassing cg07078225).

Optionally, the measuring step (b) comprises measuring a methyl group of a nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr9:127,265,221-127,265,590 (encompassing cg13912311); chr16:31,484,429-31,484,901 (encompassing cg23910243); chr20:58,180,486-58,180,815 (encompassing cg15712559); chr10:30,726,381-30,726,735 (encompassing cg04453471); chr2:66,918,184-66,918,501 (encompassing cg11469908); chr19:58,220,672-58,220,980 (encompassing cg01268824); and chr12:52,652,239-52,652,588 (encompassing cg07078225).

Optionally, the measuring step (b) comprises measuring a methyl group of a nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr9:127,265,221-127,265,590 (encompassing cg13912311); chr16:31,484,429-31,484,901 (encompassing cg23910243); chr20:58,180,486-58,180,815 (encompassing cg15712559); chr2:66,918,184-66,918,501 (encompassing cg11469908); chr19:58,220,672-58,220,980 (encompassing cg01268824); and chr12:52,652,239-52,652,588 (encompassing cg07078225).

Optionally, the measuring step (b) comprises measuring a methyl group of a nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr16:31,484,429-31,484,901 (encompassing cg23910243); chr19:58,220,672-58,220,980 (encompassing cg01268824); and chr12:52,652,239-52,652,588 (encompassing cg07078225).

Optionally, the measuring step (b) comprises measuring a methyl group of a nucleotide located at one or more of chr8:99,961,381-99,961,763 (encompassing cg08202494); chr1:170,638,675-170,639,000 (encompassing cg09010107); chr6:10,422,232-10,422,455 (encompassing cg05224741); and chr12:52,652,239-52,652,588 (encompassing cg07078225).

Optionally, the measuring step (b) comprises measuring a methyl group of a nucleotide located at one or more of chr8:99,961,381-99,961,763 (encompassing cg08202494); chr1:170,638,675-170,639,000 (encompassing cg09010107); chr6:10,422,232-10,422,455 (encompassing cg05224741); and chr12:52,652,239-52,652,588 (encompassing cg07078225).

Optionally, the measuring step (b) comprises measuring a methyl group of a nucleotide located at one or more of chr8:99,961,381-99,961,763 (encompassing cg08202494); chr6:10,422,232-10,422,455 (encompassing cg05224741); and chr12:52,652,239-52,652,588 (encompassing cg07078225).

Preferably, the measuring step (b) comprises measuring a methyl group of a nucleotide located at chr12:52,652,239-52,652,588 (encompassing cg07078225).

Optionally or additionally, the measuring step (b) comprises measuring a methyl group of a nucleotide located at one or more of chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a nucleotide located at one or more of: chr8:99,961,381-99,961,763 (encompassing cg08202494); chr15:95,836,182-95,836,449 (encompassing cg01657761); chr16:88,496,985-88,497,220 (encompassing cg03035213); chr10:30,726,381-30,726,735 (encompassing cg04453471); chr16:88,496,945-88,497,180 (encompassing cg07215504); chr2:66,918,184-66,918,501 (encompassing cg11469908); chr20:58,180,486-58,180,815 (encompassing cg15712559); chr7:47,515,002-47,515,209 (encompassing cg16329896); chr6:10,422,232-10,422,455 (encompassing cg05224741); chr1:170,638,675-170,639,000 (encompassing cg09010107); chr9:127,265,620-127,266,359 (encompassing cg04043571); chr1:197,888,169-197,888,821 (encompassing cg08610862); chr9:127,265,221-127,265,590 (encompassing cg13912311); chr8:30,244,794-30,245,560 (encompassing cg23044884); chr8:38,627,724-38,628,106 (encompassing cg14284618); chr11:6,597,801-6,598,507 (encompassing cg15511120); chr16:31,484,429-31,484,901 (encompassing cg23910243); chr19:13,616,758-13,617,066 (encompassing cg22187630); chr19:58,220,672-58,220,980 (encompassing cg01268824); chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr9:127,265,221-127,265,590 (encompassing cg13912311); chr16:31,484,429-31,484,901 (encompassing cg23910243); chr20:58,180,486-58,180,815 (encompassing cg15712559); chr10:30,726,381-30,726,735 (encompassing cg04453471); chr2:66,918,184-66,918,501 (encompassing cg11469908); chr19:58,220,672-58,220,980 (encompassing cg01268824); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr9:127,265,221-127,265,590 (encompassing cg13912311); chr16:31,484,429-31,484,901 (encompassing cg23910243); chr20:58,180,486-58,180,815 (encompassing cg15712559); chr2:66,918,184-66,918,501 (encompassing cg11469908); chr19:58,220,672-58,220,980 (encompassing cg01268824); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr16:31,484,429-31,484,901 (encompassing cg23910243); chr19:58,220,672-58,220,980 (encompassing cg01268824); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a nucleotide located at one or more of chr8:99,961,381-99,961,763 (encompassing cg08202494); chr1:170,638,675-170,639,000 (encompassing cg09010107); chr6:10,422,232-10,422,455 (encompassing cg05224741); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a nucleotide located at one or more of chr8:99,961,381-99,961,763 (encompassing cg08202494); chr1:170,638,675-170,639,000 (encompassing cg09010107); chr6:10,422,232-10,422,455 (encompassing cg05224741); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally or additionally, the measuring step (b) comprises measuring a methyl group of a nucleotide located at one or more of chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a nucleotide located at one or more of: chr8:99,961,381-99,961,763 (encompassing cg08202494); chr15:95,836,182-95,836,449 (encompassing cg01657761); chr16:88,496,985-88,497,220 (encompassing cg03035213); chr10:30,726,381-30,726,735 (encompassing cg04453471); chr16:88,496,945-88,497,180 (encompassing cg07215504); chr2:66,918,184-66,918,501 (encompassing cg11469908); chr20:58,180,486-58,180,815 (encompassing cg15712559); chr7:47,515,002-47,515,209 (encompassing cg16329896); chr6:10,422,232-10,422,455 (encompassing cg05224741); chr1:170,638,675-170,639,000 (encompassing cg09010107); chr9:127,265,620-127,266,359 (encompassing cg04043571); chr1:197,888,169-197,888,821 (encompassing cg08610862); chr9:127,265,221-127,265,590 (encompassing cg13912311); chr8:30,244,794-30,245,560 (encompassing cg23044884); chr8:38,627,724-38,628,106 (encompassing cg14284618); chr11:6,597,801-6,598,507 (encompassing cg15511120); chr16:31,484,429-31,484,901 (encompassing cg23910243); chr19:13,616,758-13,617,066 (encompassing cg22187630); chr19:58,220,672-58,220,980 (encompassing cg01268824); chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr9:127,265,221-127,265,590 (encompassing cg13912311); chr16:31,484,429-31,484,901 (encompassing cg23910243); chr20:58,180,486-58,180,815 (encompassing cg15712559); chr10:30,726,381-30,726,735 (encompassing cg04453471); chr2:66,918,184-66,918,501 (encompassing cg11469908); chr19:58,220,672-58,220,980 (encompassing cg01268824); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr9:127,265,221-127,265,590 (encompassing cg13912311); chr16:31,484,429-31,484,901 (encompassing cg23910243); chr20:58,180,486-58,180,815 (encompassing cg15712559); chr2:66,918,184-66,918,501 (encompassing cg11469908); chr19:58,220,672-58,220,980 (encompassing cg01268824); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr16:31,484,429-31,484,901 (encompassing cg23910243); chr19:58,220,672-58,220,980 (encompassing cg01268824); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a nucleotide located at one or more of chr8:99,961,381-99,961,763 (encompassing cg08202494); chr1:170,638,675-170,639,000 (encompassing cg09010107); chr6:10,422,232-10,422,455 (encompassing cg05224741); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a nucleotide located at one or more of chr8:99,961,381-99,961,763 (encompassing cg08202494); chr1:170,638,675-170,639,000 (encompassing cg09010107); chr6:10,422,232-10,422,455 (encompassing cg05224741); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a nucleotide located at one or more of chr8:99,961,381-99,961,763 (encompassing cg08202494); and chr1:170,638,675-170,639,000 (encompassing cg09010107); chr6:10,422,232-10,422,455 (encompassing cg05224741); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a cytosine nucleotide located at one or more of: chr8:99,961,381-99,961,763 (encompassing cg08202494); chr15:95,836,182-95,836,449 (encompassing cg01657761); chr16:88,496,985-88,497,220 (encompassing cg03035213); chr10:30,726,381-30,726,735 (encompassing cg04453471); chr16:88,496,945-88,497,180 (encompassing cg07215504); chr2:66,918,184-66,918,501 (encompassing cg11469908); chr20:58,180,486-58,180,815 (encompassing cg15712559); chr7:47,515,002-47,515,209 (encompassing cg16329896); chr6:10,422,232-10,422,455 (encompassing cg05224741); chr1:170,638,675-170,639,000 (encompassing cg09010107); chr9:127,265,620-127,266,359 (encompassing cg04043571); chr1:197,888,169-197,888,821 (encompassing cg08610862); chr9:127,265,221-127,265,590 (encompassing cg13912311); chr8:30,244,794-30,245,560 (encompassing cg23044884); chr8:38,627,724-38,628,106 (encompassing cg14284618); chr11:6,597,801-6,598,507 (encompassing cg15511120); chr16:31,484,429-31,484,901 (encompassing cg23910243); chr19:13,616,758-13,617,066 (encompassing cg22187630); chr19:58,220,672-58,220,980 (encompassing cg01268824); chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a cytosine nucleotide located at one or more of: chr8:99,961,381-99,961,763 (encompassing cg08202494); chr15:95,836,182-95,836,449 (encompassing cg01657761); chr16:88,496,985-88,497,220 (encompassing cg03035213); chr10:30,726,381-30,726,735 (encompassing cg04453471); chr16:88,496,945-88,497,180 (encompassing cg07215504); chr2:66,918,184-66,918,501 (encompassing cg11469908); chr20:58,180,486-58,180,815 (encompassing cg15712559); chr7:47,515,002-47,515,209 (encompassing cg16329896); chr6:10,422,232-10,422,455 (encompassing cg05224741); chr1:170,638,675-170,639,000 (encompassing cg09010107); chr9:127,265,620-127,266,359 (encompassing cg04043571); chr1:197,888,169-197,888,821 (encompassing cg08610862); chr9:127,265,221-127,265,590 (encompassing cg13912311); chr8:30,244,794-30,245,560 (encompassing cg23044884); chr8:38,627,724-38,628,106 (encompassing cg14284618); chr11:6,597,801-6,598,507 (encompassing cg15511120); chr16:31,484,429-31,484,901 (encompassing cg23910243); chr19:13,616,758-13,617,066 (encompassing cg22187630); chr19:58,220,672-58,220,980 (encompassing cg01268824); and chr12:52,652,239-52,652,588 (encompassing cg07078225).

Optionally, the measuring step (b) comprises measuring a methyl group of a cytosine nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr9:127,265,221-127,265,590 encompassing cg13912311; chr16:31,484,429-31,484,901 (encompassing cg23910243); chr20:58,180,486-58,180,815 (encompassing cg15712559); chr10:30,726,381-30,726,735 (encompassing cg04453471); chr2:66,918,184-66,918,501 (encompassing cg11469908); chr19:58,220,672-58,220,980 (encompassing cg01268824); and chr12:52,652,239-52,652,588 (encompassing cg07078225).

Optionally, the measuring step (b) comprises measuring a methyl group of a cytosine nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr16:31,484,429-31,484,901 (encompassing cg23910243); and chr12:52,652,239-52,652,588 (encompassing cg07078225).

Optionally, the measuring step (b) comprises measuring a methyl group of a cytosine nucleotide located at one or more of chr8:99,961,381-99,961,763 (encompassing cg08202494); chr1:170,638,675-170,639,000 (encompassing cg09010107); chr6:10,422,232-10,422,455 (encompassing cg05224741); and chr12:52,652,239-52,652,588 (encompassing cg07078225).

Optionally, the measuring step (b) comprises measuring a methyl group of a cytosine nucleotide located at one or more of chr8:99,961,381-99,961,763 (encompassing cg08202494); chr1:170,638,675-170,639,000 (encompassing cg09010107); and chr12:52,652,239-52,652,588 (encompassing cg07078225).

Optionally, the measuring step (b) comprises measuring a methyl group of a cytosine nucleotide located at one or more of chr8:99,961,381-99,961,763 (encompassing cg08202494); and chr6:10,422,232-10,422,455 (encompassing cg05224741); and chr12:52,652,239-52,652,588 (encompassing cg07078225).

Optionally, the measuring step (b) comprises measuring a methyl group of a cytosine nucleotide located at one or more of chr8:99,961,381-99,961,763 (encompassing cg08202494); chr6:10,422,232-10,422,455 (encompassing cg05224741); and chr12:52,652,239-52,652,588 (encompassing cg07078225).

Optionally, the measuring step (b) comprises measuring a methyl group of a cytosine nucleotide located at chr6:10,422,232-10,422,455 (encompassing cg05224741); and chr12:52,652,239-52,652,588 (encompassing cg07078225).

Preferably, the measuring step (b) comprises measuring a methyl group of a cytosine nucleotide located at chr12:52,652,239-52,652,588 (encompassing cg07078225).

Optionally or additionally, the measuring step (b) comprises measuring a methyl group of a cytosine nucleotide located at one or more of chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a cytosine nucleotide located at one or more of: chr8:99,961,381-99,961,763 (encompassing cg08202494); chr15:95,836,182-95,836,449 (encompassing cg01657761); chr16:88,496,985-88,497,220 (encompassing cg03035213); chr10:30,726,381-30,726,735 (encompassing cg04453471); chr16:88,496,945-88,497,180 (encompassing cg07215504); chr2:66,918,184-66,918,501 (encompassing cg11469908); chr20:58,180,486-58,180,815 (encompassing cg15712559); chr7:47,515,002-47,515,209 (encompassing cg16329896); chr6:10,422,232-10,422,455 (encompassing cg05224741); chr1:170,638,675-170,639,000 (encompassing cg09010107); chr9:127,265,620-127,266,359 (encompassing cg04043571); chr1:197,888,169-197,888,821 (encompassing cg08610862); chr9:127,265,221-127,265,590 (encompassing cg13912311); chr8:30,244,794-30,245,560 (encompassing cg23044884); chr8:38,627,724-38,628,106 (encompassing cg14284618); chr11:6,597,801-6,598,507 (encompassing cg15511120); chr16:31,484,429-31,484,901 (encompassing cg23910243); chr19:13,616,758-13,617,066 (encompassing cg22187630); chr19:58,220,672-58,220,980 (encompassing cg01268824); chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a cytosine nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr9:127,265,221-127,265,590 (encompassing cg13912311); chr16:31,484,429-31,484,901 (encompassing cg23910243); chr20:58,180,486-58,180,815 (encompassing cg15712559); chr10:30,726,381-30,726,735 (encompassing cg04453471); chr2:66,918,184-66,918,501 (encompassing cg11469908); chr19:58,220,672-58,220,980 (encompassing cg01268824); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a cytosine nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr9:127,265,221-127,265,590 (encompassing cg13912311); chr16:31,484,429-31,484,901 (encompassing cg23910243); chr20:58,180,486-58,180,815 (encompassing cg15712559); chr2:66,918,184-66,918,501 (encompassing cg11469908); chr19:58,220,672-58,220,980 (encompassing cg01268824); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a cytosine nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr16:31,484,429-31,484,901 (encompassing cg23910243); chr19:58,220,672-58,220,980 (encompassing cg01268824); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a cytosine nucleotide located at one or more of chr8:99,961,381-99,961,763 (encompassing cg08202494); chr1:170,638,675-170,639,000 (encompassing cg09010107); chr6:10,422,232-10,422,455 (encompassing cg05224741); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a cytosine nucleotide located at one or more of chr8:99,961,381-99,961,763 (encompassing cg08202494); chr1:170,638,675-170,639,000 (encompassing cg09010107); chr6:10,422,232-10,422,455 (encompassing cg05224741); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally, the measuring step (b) comprises measuring a methyl group of a cytosine nucleotide located at one or more of chr12:52,652,239-52,652,588 (encompassing cg07078225); chr8:99,961,381-99,961,763 (encompassing cg08202494); and chr1:170,638,675-170,639,000 (encompassing cg09010107); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

Optionally or additionally, the measuring step (b) comprises measuring a methyl group of a cytosine nucleotide located at one or more of chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a cytosine nucleotide located at one or more of chr8:99,961,381-99,961,763 (encompassing cg08202494); chr15:95,836,182-95,836,449 (encompassing cg01657761); chr16:88,496,985-88,497,220 (encompassing cg03035213); chr10:30,726,381-30,726,735 (encompassing cg04453471); chr16:88,496,945-88,497,180 (encompassing cg07215504); chr2:66,918,184-66,918,501 (encompassing cg11469908); chr20:58,180,486-58,180,815 (encompassing cg15712559); chr7:47,515,002-47,515,209 (encompassing cg16329896); chr6:10,422,232-10,422,455 (encompassing cg05224741); chr1:170,638,675-170,639,000 (encompassing cg09010107); chr9:127,265,620-127,266,359 (encompassing cg04043571); chr1:197,888,169-197,888,821 (encompassing cg08610862); chr9:127,265,221-127,265,590 (encompassing cg13912311); chr8:30,244,794-30,245,560 (encompassing cg23044884); chr8:38,627,724-38,628,106 (encompassing cg14284618); chr11:6,597,801-6,598,507 (encompassing cg15511120); chr16:31,484,429-31,484,901 (encompassing cg23910243); chr19:13,616,758-13,617,066 (encompassing cg22187630); chr19:58,220,672-58,220,980 (encompassing cg01268824); chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a cytosine nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr9:127,265,221-127,265,590 (encompassing cg13912311); chr16:31,484,429-31,484,901 (encompassing cg23910243); chr20:58,180,486-58,180,815 (encompassing cg15712559); chr10:30,726,381-30,726,735 (encompassing cg04453471); chr2:66,918,184-66,918,501 (encompassing cg11469908); chr19:58,220,672-58,220,980 (encompassing cg01268824); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a cytosine nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr9:127,265,221-127,265,590 (encompassing cg13912311); chr16:31,484,429-31,484,901 (encompassing cg23910243); chr20:58,180,486-58,180,815 (encompassing cg15712559); chr2:66,918,184-66,918,501 (encompassing cg11469908); chr19:58,220,672-58,220,980 (encompassing cg01268824); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a cytosine nucleotide located at one or more of chr8:99,961,381-99,961,763 (encompassing cg08202494); chr6:10,422,232-10,422,455 (encompassing cg05224741); chr1:170,638,675-170,639,000 (encompassing cg09010107); chr16:31,484,429-31,484,901 (encompassing cg23910243); chr19:58,220,672-58,220,980 (encompassing cg01268824); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a cytosine nucleotide located at one or more of chr8:99,961,381-99,961,763 (encompassing cg08202494); chr1:170,638,675-170,639,000 (encompassing cg09010107); chr6:10,422,232-10,422,455 (encompassing cg05224741); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a cytosine nucleotide located at one or more of chr8:99,961,381-99,961,763 (encompassing cg08202494); chr1:170,638,675-170,639,000 (encompassing cg09010107); chr6:10,422,232-10,422,455 (encompassing cg05224741); and chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the measuring step (b) comprises measuring a methyl group of a cytosine nucleotide located at one or more of chr12:52,652,239-52,652,588 (encompassing cg07078225); chr8:99,961,381-99,961,763 (encompassing cg08202494); and chr1:170,638,675-170,639,000 (encompassing cg09010107); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328).

Optionally, the predicting step comprises comparing the nucleic acid methylation level of the or each biomarker with the nucleic acid methylation level of a respective normal.

Optionally, the respective normal is the respective biomarker in a patient not suffering from ovarian cancer. Further optionally, the nucleic acid methylation level of the respective normal is the nucleic acid methylation level of the respective biomarker in a patient not suffering from ovarian cancer.

Optionally, the respective normal is the respective biomarker in a sample from a patient not suffering from ovarian cancer. Further optionally, the nucleic acid methylation level of the respective normal is the nucleic acid methylation level of the respective biomarker in a sample from a patient not suffering from ovarian cancer.

Optionally, the respective normal is the respective biomarker in a tissue sample from a patient not suffering from ovarian cancer. Further optionally, the nucleic acid methylation level of the respective normal is the nucleic acid methylation level of the respective biomarker in a tissue sample from a patient not suffering from ovarian cancer.

Optionally, the respective normal is the respective biomarker in an epithelial tissue sample from a patient not suffering from ovarian cancer. Further optionally, the nucleic acid methylation level of the respective normal is the nucleic acid methylation level of the respective biomarker in an epithelial tissue sample from a patient not suffering from ovarian cancer.

Optionally, the respective normal is the respective biomarker in an epithelial tissue sample from a fallopian tube or ovary of a patient not suffering from ovarian cancer. Further optionally, the nucleic acid methylation level of the respective normal is the nucleic acid methylation level of the respective biomarker in an epithelial tissue sample from a fallopian tube or ovary of a patient not suffering from ovarian cancer. Further optionally, the nucleic acid methylation level of the respective normal is the nucleic acid methylation level of the respective biomarker levels in blood and/or plasma from a patient not suffering from ovarian cancer.

Optionally, deviation of the nucleic acid methylation level of the or each biomarker from the nucleic acid methylation level of the respective normal is indicative of ovarian cancer. Further optionally, deviation of the nucleic acid methylation level of all biomarkers from the nucleic acid methylation level of the respective normals is indicative of ovarian cancer.

Optionally, a nucleic acid methylation level of the or each biomarker higher than the nucleic acid methylation level of the respective normal is indicative of ovarian cancer.

Optionally, a nucleic acid methylation level of the or each biomarker higher than the nucleic acid methylation level of the respective normal is indicative of serous tubal intraepithelial carcinoma.

Optionally, a nucleic acid methylation level of the or each biomarker higher than the nucleic acid methylation level of the respective normal is indicative of high grade serous carcinoma.

Optionally, a nucleic acid methylation level of the or each biomarker higher than the nucleic acid methylation level of the respective normal is indicative of fallopian tube cancer.

Optionally, a nucleic acid methylation level of the or each biomarker higher than a threshold value is indicative of ovarian cancer.

Further optionally, a nucleic acid methylation level of the or each biomarker higher than a threshold value is indicative of ovarian cancer, wherein the respective threshold value of the or each biomarker is:

| Biomarker | Threshold value |
|---|---|
| cg08202494 | 0.39 |
| cg03035213 | 0.12 |
| cg03314029 | 0.20 |
| cg04043571 | 0.21 |
| cg04453471 | 0.20 |
| cg07215504 | 0.11 |
| cg11469908 | 0.29 |
| cg15712559 | 0.17 |
| cg16329896 | 0.20 |
| cg05224741 | 0.06 |
| cg09010107 | 0.40 |
| cg24376434 | 0.45 |
| cg08610862 | 0.31 |
| cg13912311 | 0.25 |
| cg23044884 | 0.38 |
| cg20343048 | 0.39 |
| cg15511120 | 0.36 |
| cg23910243 | 0.46 |
| cg14284618 | 0.33 |
| cg22187630 | 0.19 |
| cg07078225 | 0.25 |
| cg02766845 | 0.12 |
| cg03109827 | 0.12 |
| cg07489502 | 0.18 |
| cg07316846 | 0.12 |
| cg08447324 | 0.09 |
| cg08958294 | 0.10 |
| cg17754510 | 0.13 |
| cg18617005 | 0.12 |
| cg19200589 | 0.14 |
| cg26365299 | 0.15 |
| cg03692651 | 0.17 |
| cg07907386 | 0.12 |
| cg08189989 | 0.11 |
| cg10293403 | 0.14 |
| cg11528328 | 0.26 |
| cg12595013 | 0.15 |
| cg20935165 | 0.11 |
| cg23200020 | 0.12 |
| cg23290344 | 0.14 |
| cg24884703 | 0.16 |
| cg25622366 | 0.15 |

Optionally, a nucleic acid methylation level of the or each biomarker higher than a threshold value is indicative of fallopian tube cancer.

Further optionally, a nucleic acid methylation level of the or each biomarker higher than a threshold value is indicative of fallopian tube cancer, wherein the respective threshold value of the or each biomarker is:

| Biomarker | Threshold value |
|---|---|
| cg08202494 | 0.39 |
| cg03035213 | 0.12 |
| cg03314029 | 0.20 |
| cg04043571 | 0.21 |
| cg04453471 | 0.20 |
| cg07215504 | 0.11 |
| cg11469908 | 0.29 |
| cg15712559 | 0.17 |
| cg16329896 | 0.20 |
| cg05224741 | 0.06 |
| cg09010107 | 0.40 |
| cg24376434 | 0.45 |
| cg08610862 | 0.31 |
| cg13912311 | 0.25 |
| cg23044884 | 0.38 |
| cg20343048 | 0.39 |
| cg15511120 | 0.36 |
| cg23910243 | 0.46 |
| cg14284618 | 0.33 |
| cg22187630 | 0.19 |
| cg07078225 | 0.25 |
| cg02766845 | 0.12 |
| cg03109827 | 0.12 |
| cg07489502 | 0.18 |
| cg07316846 | 0.12 |
| cg08447324 | 0.09 |
| cg08958294 | 0.10 |
| cg17754510 | 0.13 |
| cg18617005 | 0.12 |
| cg19200589 | 0.14 |
| cg26365299 | 0.15 |
| cg03692651 | 0.17 |
| cg07907386 | 0.12 |
| cg08189989 | 0.11 |
| cg10293403 | 0.14 |
| cg11528328 | 0.26 |
| cg12595013 | 0.15 |
| cg20935165 | 0.11 |
| cg23200020 | 0.12 |
| cg23290344 | 0.14 |
| cg24884703 | 0.16 |
| cg25622366 | 0.15 |

Optionally, a nucleic acid methylation level of the or each biomarker higher than a threshold value is indicative of serous tubal intraepithelial carcinoma.

Further optionally, a nucleic acid methylation level of the or each biomarker higher than a threshold value is indicative of serous tubal intraepithelial carcinoma, wherein the respective threshold value of the or each biomarker is:

| Biomarker | Threshold value |
|---|---|
| cg08202494 | 0.39 |
| cg03035213 | 0.12 |
| cg03314029 | 0.20 |
| cg04043571 | 0.21 |
| cg04453471 | 0.20 |
| cg07215504 | 0.11 |
| cg11469908 | 0.29 |
| cg15712559 | 0.17 |
| cg16329896 | 0.20 |
| cg05224741 | 0.06 |
| cg09010107 | 0.40 |
| cg24376434 | 0.45 |
| cg08610862 | 0.31 |
| cg13912311 | 0.25 |
| cg23044884 | 0.38 |
| cg20343048 | 0.39 |
| cg15511120 | 0.36 |
| cg23910243 | 0.46 |
| cg14284618 | 0.33 |
| cg22187630 | 0.19 |
| cg07078225 | 0.25 |
| cg02766845 | 0.12 |
| cg03109827 | 0.12 |
| cg07489502 | 0.18 |
| cg07316846 | 0.12 |
| cg08447324 | 0.09 |
| cg08958294 | 0.10 |
| cg17754510 | 0.13 |
| cg18617005 | 0.12 |
| cg19200589 | 0.14 |
| cg26365299 | 0.15 |
| cg03692651 | 0.17 |
| cg07907386 | 0.12 |
| cg08189989 | 0.11 |
| cg10293403 | 0.14 |
| cg11528328 | 0.26 |
| cg12595013 | 0.15 |
| cg20935165 | 0.11 |
| cg23200020 | 0.12 |
| cg23290344 | 0.14 |
| cg24884703 | 0.16 |
| cg25622366 | 0.15 |

Optionally, a nucleic acid methylation level of the or each biomarker higher than a threshold value is indicative of high grade serous carcinoma.

Further optionally, a nucleic acid methylation level of the or each biomarker higher than a threshold value is indicative of high grade serous carcinoma, wherein the respective threshold value of the or each biomarker is:

| Biomarker | Threshold value |
|---|---|
| cg08202494 | 0.51 |
| cg03035213 | 0.20 |
| cg03314029 | 0.21 |
| cg04043571 | 0.26 |
| cg04453471 | 0.31 |
| cg07215504 | 0.16 |
| cg11469908 | 0.48 |
| cg15712559 | 0.17 |
| cg16329896 | 0.55 |
| cg05224741 | 0.48 |
| cg09010107 | 0.53 |
| cg24376434 | 0.62 |
| cg08610862 | 0.47 |
| cg13912311 | 0.40 |
| cg23044884 | 0.54 |
| cg20343048 | 0.53 |
| cg15511120 | 0.48 |
| cg23910243 | 0.62 |
| cg14284618 | 0.46 |
| cg22187630 | 0.26 |
| cg07078225 | 0.57 |
| cg02766845 | 0.39 |
| cg03109827 | 0.36 |
| cg07489502 | 0.52 |
| cg07316846 | 0.48 |
| cg08447324 | 0.31 |
| cg08958294 | 0.38 |
| cg17754510 | 0.60 |
| cg18617005 | 0.47 |
| cg19200589 | 0.41 |
| cg26365299 | 0.38 |
| cg03692651 | 0.47 |
| cg07907386 | 0.35 |
| cg08189989 | 0.38 |
| cg10293403 | 0.43 |
| cg11528328 | 0.51 |
| cg12595013 | 0.47 |
| cg20935165 | 0.45 |
| cg23200020 | 0.45 |
| cg23290344 | 0.49 |
| cg24884703 | 0.48 |
| cg25622366 | 0.55 |

Optionally, the biological sample is selected from tissue, whole blood, serum, plasma, urine, interstitial fluid, peritoneal fluid, cervical sampling, tears, saliva, pleural fluid, intra-uterine lavage/fluid, vaginal lavage/fluid, and cerebrospinal fluid.

Further optionally, the biological sample is selected from whole blood, serum, and plasma.

Preferably, the biological sample is plasma.

Optionally, the providing step (a) comprises the step of providing a nucleic acid sample from the patient. Further optionally, the providing step (a) comprises the step of providing a nucleic acid sample from the biological sample. Still further optionally, the providing step (a) comprises the step of providing a nucleic acid sample from the whole blood, serum, and/or plasma sample.

Optionally, the providing step (a) comprises the step of providing a deoxyribonucleic acid sample from the patient. Further optionally, the providing step (a) comprises the step of providing a deoxyribonucleic acid sample from the biological sample. Still further optionally, the providing step (a) comprises the step of providing a deoxyribonucleic acid sample from the whole blood, serum, and/or plasma sample.

Optionally, the providing step (a) comprises the step of isolating a deoxyribonucleic acid sample from the patient. Further optionally, the providing step (a) comprises the step of isolating a deoxyribonucleic acid sample from the biological sample. Still further optionally, the providing step (a) comprises the step of isolating a deoxyribonucleic acid sample from the whole blood sample, serum, and plasma.

Optionally, the patient is a human.

Optionally, the patient is a female. Further optionally, the patient is a female human.

Optionally, the method is a method for identifying ovarian cancer in a patient, and the method comprises the steps of: providing a biological sample from the patient; measuring the nucleic acid methylation levels of one or more biomarkers in the sample; and identifying ovarian cancer in the patient based on the nucleic acid methylation levels.

Optionally, the method is a method for predicting ovarian cancer in a patient, and the method comprises the steps of: providing a biological sample from the patient; measuring the nucleic acid methylation levels of one or more biomarkers in the sample; and predicting ovarian cancer in the patient based on the nucleic acid methylation levels.

Optionally, the method is a method for diagnosing serous tubal intraepithelial carcinoma in a patient, and the method comprises the steps of: providing a biological sample from the patient; measuring the nucleic acid methylation levels of one or more biomarkers in the sample; and diagnosing serous tubal intraepithelial carcinoma in the patient based on the nucleic acid methylation levels.

Optionally, the method is a method for diagnosing high grade serous carcinoma in a patient, and the method comprises the steps of: providing a biological sample from the patient; measuring the nucleic acid methylation levels of one or more biomarkers in the sample; and diagnosing high grade serous carcinoma in the patient based on the nucleic acid methylation levels.

Optionally, the method is a method for diagnosing fallopian tube cancer in a patient, and the method comprises the steps of: providing a biological sample from the patient; measuring the nucleic acid methylation levels of one or more biomarkers in the sample; and diagnosing fallopian tube cancer in the patient based on the nucleic acid methylation levels.

According to a further aspect of the present invention, there is provided a method of monitoring ovarian cancer in a patient, the method comprising the steps of:
(a) providing a first biological sample from the patient;
(b) measuring the nucleic acid methylation levels of one or more biomarkers in the first sample;
(c) providing a second or subsequent biological sample from the patient;
(d) measuring the nucleic acid methylation levels of one or more biomarkers in the second or subsequent sample; and
(e) comparing the nucleic acid methylation levels of the one or more biomarkers in the first sample with the nucleic acid methylation levels of one or more biomarkers in the second or subsequent sample.

Optionally, deviation of the nucleic acid methylation level of the or each biomarker in the second or subsequent sample from the nucleic acid methylation level of the or each biomarker in the first sample is indicative of progression of ovarian cancer. Further optionally, deviation of the nucleic acid methylation level of all biomarkers in the second or subsequent sample from the nucleic acid methylation level of the or each biomarker in the first sample is indicative of progression of ovarian cancer.

Optionally, a nucleic acid methylation level of the or each biomarker in the second or subsequent sample higher than the nucleic acid methylation level of the or each biomarker in the first sample is indicative of progression of ovarian cancer.

Optionally, a nucleic acid methylation level of the or each biomarker in the second or subsequent sample higher than the nucleic acid methylation level of the or each biomarker in the first sample is indicative of serous tubal intraepithelial carcinoma.

Optionally, a nucleic acid methylation level of the or each biomarker in the second or subsequent sample higher than the nucleic acid methylation level of the or each biomarker in the first sample is indicative of high grade serous carcinoma.

Optionally, a nucleic acid methylation level of the or each biomarker in the second or subsequent sample higher than the nucleic acid methylation level of the or each biomarker in the first sample is indicative of fallopian tube cancer.

According to a still further aspect of the present invention, there is provided an assay for diagnosing and/or monitoring ovarian cancer in the patient, the assay comprising means for measuring the nucleic acid methylation levels of the one or more biomarkers in the biological sample from the patient.

According to a still further aspect of the present invention, there is provided an assay for prognosing and/or monitoring ovarian cancer in the patient, the assay comprising means for measuring the nucleic acid methylation levels of the one or more biomarkers in the biological sample from the patient.

Optionally, the assay further comprises means for receiving the biological sample from the patient.

### BRIEF DESCRIPTION OF THE DRAWINGS

Embodiments of the present invention will now be described with reference to the following non-limiting examples and the accompanying drawings in which:

FIG. 1 is a summary diagram of patient numbers and tissue type included in pilot, validation, and longitudinal blood sample collection cohorts;

FIG. 2 is scatter plots showing DNA methylation levels of candidate differentially methylated regions (DMRs) in whole blood samples from healthy individuals according to (A) GSE41169 and (B) GSE123914 datasets, wherein the beta value on the y-axis indicates the DNA methylation level, wherein DNA methylation increases from 0 to 1, and wherein candidate DMRs are labelled on the x-axis;

FIG. 3 illustrates methylation scores (%) in normal fallopian tube (NFT), STIC and HGSC formalin-fixed paraffin-embedded (FFPE) tissue samples for nine DNA methylation markers, wherein data were analysed between groups (NFT-STIC, STIC-HGSC, NFT-HGSC) using the Mann-Whitney U test and presented as median (IQR);

FIG. 4 is scatter plots for seven DNAme markers showing statistically significant hypermethylation in a HGSC group compared to an NFT group (both n=48) following pyrosequencing analysis of a validation cohort (p<0.0001 for all seven DNAme markers);

FIG. 5 illustrates receiver operating characteristic (ROC) curves for CA125 and 7 selected DNA methylation markers following pyrosequencing analysis in a validation cohort (48 non-cancerous NFT and 48 HGSC FFPE tissue samples); wherein three DNAme markers cg08202494, cg09010107 and cg11469908 achieved improved diagnostic accuracy compared to CA125; wherein methylation scores were available for all 48 of the NFT group and all 48 of the HGSC group for DNAme markers cg09010107, cg13912311, cg23910243, cg04453471_B and cg11469908; and wherein methylation scores for 45 of the NFT and 45 of the HGSC group were available for the DNAme marker cg08202494; and wherein CA125 values were available for 18 out of 48 of the NFT group and all 48 of the HGSC group;

FIG. 6 illustrates methylation scores (%) for the same 7 selected markers as FIG. 5, comparing NFT with HGSC (n=48) and showing breakdown of all International Federation of Gynecology and Obstetrics (FIGO) stages in the HGSC cohort;

FIG. 7 illustrates DNA electrophoresis gel showing PCR product following digestion with: HpaII/AciI; wherein lane 1 is cg08202494 methylated control, lane 2 is cg08202494 control, lane 3 is cg09010107 methylated control, lane 4 is cg09010107 unmethylated control, lane 5 is ACTB methylated control, and lane 6 is ACTB unmethylated control;

FIG. 8 illustrates DNA electrophoresis gel showing PCR product for variable genomic DNA concentration inputs with 10 ng (lane 1), 1 ng (lane 2), 0.1 ng (lane 3) and 0.01 ng (lane 4), NTC (lane 5), and undigested 10 ng genomic DNA (lane 6);

FIG. 9 illustrates a representative calibration curve using 10 ng input DNA;

FIG. 10 illustrates a scatter plot of MSRE-q PCR for (A) cg08202494 and (B) cg09010107 relative to ACTB reference control, wherein cg08202494 and cg09010107 methylation status was determined in 48 NFT and 48 HGSC tissue samples by MSRE qPCR; wherein statistically significant hypermethylation (p<0.0001) was observed in a HGSC cohort compared to a NFT cohort in both markers, wherein the bars represent the median value with interquartile range, wherein p values were calculated by Mann-Whitney U test and p<0.05 was considered statistically significant;

FIG. 11 illustrates ROC analysis of (A) cg08202494 and (B) cg09010107 performance in 48 NFT and 48 HGSC tissue samples, wherein cg08202494 and cg09010107 show improved diagnostic accuracy compared to CA125 in this cohort, wherein cg08202494 achieved an AUC of 0.9461 (95% CI, 0.8881-1.004) and cg09010107 achieved an AUC of 0.9396 (95 5 CI, 0.8906-0.9885) compared to an AUC of 0.912 (95% CI, 0.805-1.0) for CA125;

FIG. 12 is a workflow of MSRE qPCR analyses;

FIG. 13 is a scatter plot showing relative quantification of cg08202494 in matched FFPE and plasma samples; wherein data were analysed using the Mann-Whitney U test and presented as median (IQR), wherein p<0.05 was deemed as statistically significant;

FIG. 14 is a heatmap showing correlation between g08202494 matched FFPE tissue and plasma samples;

FIG. 15 illustrates ROC curve analyses of cg08202494 assay diagnostic performance in FFPE tissue and matched plasma samples;

FIGS. 16A and 16B are each a schematic process of identification and screening of DNAme markers (listed in Table 3A and 3B, respectively);

FIG. 17 illustrates DNA agarose gels showing semi-quantitative PCR reactions of MSRE digested DNA samples from M (methylated control DNA) and U (unmethylated control DNA), with N (no template control) used as negative control, wherein an additional differential positive control PCR (cg07078225, KRT87P) and PCR control (beta actin) are also shown;

FIG. 18 illustrates DNA agarose gels showing semi-quantitative PCR reactions of MSRE digested DNA samples from M (methylated control DNA) and U (unmethylated control DNA), with N (no template control) used as negative control wherein NFT 1/2/3 represents NFT FFPE from 3 separate patients and HGSC 1/2/3 represents HGSC FFPE from 3 separate patients; and

FIG. 19 illustrates bar graphs showing average methylation (Beta) values for an in-house, case matched, cohort of NFT (Normal Fallopian Tube), STIC (Serous Tubal Intraepithelial Carcinoma) and HGSC (High Grade Serous Carcinoma) FFPE samples, all measured by Illumina 450K arrays, which were compared alongside publically available methylation datasets, including datasets taken from control patient WB (Whole blood), and patients with OA (Osteoarthritis), RA (Rheumatoid Arthritis) and MS (Multiple Sclerosis), wherein datasets can be accessed via GSE123914 (WB), GSE46650 (OA and RA) and GSE88824 (MS), respectively, wherein all cg tiles show increasing methylation from NFT to STIC to HGSC and most show low baseline levels in control (WB) and inflammatory condition datasets (OA, RA, MS), indicating that they represent excellent candidates for the development of ovarian cancer diagnostic and disease monitoring markers.

### EXAMPLES

Materials and Methods

Genome Wide DNA Methylation (DNAme) Profiling

Candidate DNA methylation markers were identified in Beirne JP. “The identification and characterisation of disease-specific biomarkers in pelvic high grade serous carcinomas” 2016; wherein tissue samples from a cohort of six HGSC patients (the pilot cohort) were analysed using the Illumina® Infinium Human Methylation 450K BeadChip® array. The bioinformatic analyses carried out in Beirne JP 2016 were to identify the top ranking DNAme markers from the Illumina® array and were performed by Dr Darragh McArt, Reader in Cancer Bioinformatics at HSB-QUB and his team.

Infinium Human Methylation 450K BeadChip® arrays were performed as previously described in Beirne JP, “The identification and characterisation of disease-specific biomarkers in pelvic high grade serous carcinomas”, 2016 (available at https://ethos.bl.uk/OrderDetails.do?uin=uk.bl.ethos.705640).

Methylation profiles are available on Gene Expression Omnibus (GEO; http://www.ncbi.nlm.nih.gov/geo/; accession number: GSE41169 and GSE123914). These datasets were used as a blood control (i.e. healthy donors) for verification of tissue specificity of the loci of interest. The GSE41169 dataset (n=95 male and female participants) comprises genome wide DNA methylation profiling of whole blood in schizophrenia patients and healthy subjects of different ages. In this dataset, the Illumina® Infinium 450K Human DNA methylation Beadchip® was used to obtain DNA methylation profiles. The GSE123914 dataset (n=35 female participants) used the Illumina® Methylation EPIC (850K) Beadchip® to obtain DNA methylation profiles in paired whole blood samples collected approximately one year apart from 35 healthy women enrolled in the Nurses Study II cohort.

Methylation Marker Discovery Analysis

Probes were selected for absence of methylation in leukocytes (GSE41169 and GSE123014; maximum beta value allowed=0.2) minimising the risk of false positivity in blood tests which could be caused by methylated DNA from blood cells.

Methylation Assays for Pyrosequencing Analysis

DNAme marker sequences were identified through the Integrative Genomics Viewer (IGV, Broad Institute, Massachusetts, USA). Sequences were manipulated in-silico to reflect post-bisulphite conversion sequence changes. Sequences were imported into Pyromark Assay Design 2.0 (Qiagen, Manchester, UK) to facilitate assay design. Regions of interest were defined and optimal forward, reverse, and sequencing pyrosequencing primers were designed adhering to the manufacturer's guidelines (see Table 1).

TABLE 1
Forward, reverse, and sequencing pyrosequencing primers

| DMR | Forward primer | Reverse primers | Sequence primer |
|---|---|---|---|
| cg08202494 | 5′- | 5′-Biotin- | 5′- |
| | ATTTTATTTTAAGTAGG | TAAACTCAACTTACTCA | TTTTTAGTTTTGTATAA |
| | GGGTTATTGT-3′ | AATCTCCCATCTT-3 | GGA-3′ |
| cg01657761 | 5′- | 5′-Biotin- | 5′- |
| | TTGTGTTGTTGATGTT | ACAACTAAAATTTACAT | TGATGTTGGAATGTAG |
| | GGAATGTAG-3′ | AACTCCT-3′ | G-3′ |
| cg03035213 | 5′- | 5′-Biotin- | 5′- |
| | GGGGTAAGGAGTTTAT | AAACCTACCATCCTCA | TGAAGATAGTGTAGTA |
| | TTTGA-3′ | ACTCC-3′ | GAAGAATAG-3 |
| cg03314029 | 5′- | 5′-Biotin- | 5′- |
| | GAGGGGGAAGTAGTT | ACCTCAAACCCAAAAC | AGTTGTTTAGGAAGGA |
| | GAAG-3′ | TCTACC-3′ | TT-3′ |
| cg04453471_A | 5′-Biotin- | 5′- | 5′- |
| | AGAGGAATGTTGGATT | CCCAACCATCTACCTC | CATCATTCACTTACTTA |
| | GTAGGATGTTA-3′ | TCAATAAATTTCA-3′ | ATAATC-3′ |
| cg04453471_B | 5′- | 5′-Biotin- | 5′- |
| | TATTGAGAGGTAGATG | ATACTCCACTACAAAA | AGATGGTTGGGTTATG- |
| | GTTGGGTTAT-3 | CCACCT-3′ | 3′ |
| cg07215504 | 5′- | 5′-Biotin- | 5′- |
| | TTTAGGAAGGATTTTA | CTTCTACTACACCATCT | TAGGAAGAGGAAGGT- |
| | GGAAGAGG-3′ | TCAAAATAAACTC-3′ | 3′ |
| cg11469908 | 5′- | 5′-Biotin- | 5′- |
| | TAGTGTTTGTAAAAGA | ATAACCTTCTAAACACT | TTGTAAAAGAAGGGGA |
| | AGGGGAAAT-3 | TTCTTCCATATA-3 | AATA-3′ |
| cg15712559 | 5′- | 5′-Biotin- | 5′- |
| | GTTTGGGTATTTGTTTT | ACTCTACCCAACACAC | ATTTGTTTTTGGAAGGT |
| | TGGAAGGTA-3′ | CTATC-3′ | AG-3′ |
| cg16329896 | 5′- | 5′-Biotin- | 5′- |
| | GAGTTGGATTAGGTAT | CCTATTTACAAAAAAAC | TGTTTTGAATTTTTTTA |
| | TGTTTTGAAT-3′ | CACTATCCT-3′ | TATTAGAG-3′ |
| cg05224741 | 5′- | 5′-Biotin- | 5′- |
| | AGATTTGAGTTTTTTTT | ACTAAAAAACATCCCC | TTGTTGTATAGTTTAGA |
| | TGGTTTTTTAA-3′ | CATCC-3′ | GTTT-3′ |
| cg09010107 | 5′-Biotin- | 5′- | 5′- |
| | AAATTTTTTTTGGAGGA | AACACAAACACCTACA | CAAACCTTTACAACAAT |
| | AGTATTAGGGT-3′ | AACCTTTACAACAA-3′ | CC-3′ |
| cg04043571 | 5′- | 5′-Biotin- | 5′- |
| | TGTATATAGAAATAATT | CCCCAACCCTCCTAAA | GAAATAATTGGAAATA |
| | GGAAATAGGG-3′ | TCCCCTAA-3′ | GGGT-3′ |
| cg08610862 | 5′- | 5′-Biotin- | 5′- |
| | AGGGAAAGAGGGGAT | ACCCCTACTATTCCCT | AGAGGGGATTGTATAT |
| | TGTAT-3′ | AATTTTCAA-3′ | AG-3′ |
| cg13912311 | 5′- | 5′-Biotin- | 5′- |
| | TTTTTAGGTTGTGGGG | ATCCCTTCTACCCCTT | GTTGTGGGGGGTTAG- |
| | GGTTA-3′ | CCAAAAATACCT-3′ | 3′ |
| cg23044884 | 5′-Biotin- | 5′- | 5′- |
| | AAATTTTGGAGGTGGA | AACTCATTTTTCCATAT | ATTCAATAACTACCAAA |
| | TTTTAGTAAATAG-3′ | CAATTCAATAAC-3′ | AATTACAA-3′ |
| cg14284618 | 5′- | 5′-Biotin- | 5′- |
| | GTATGTAGATTTTTTTT | AAACATAATTCAACTCC | AGATTTTTTGTTTTTTG |
| | TAGGGGTTGTAG-3′ | CAAAAAAATTTCC-3′ | AATAATTT-3′ |
| cg15511120 | 5′- | 5′-Biotin- | 5′- |
| | GATAAGAAAGGGAAGA | CCAATAAAAAAAAAAC | ATTTGATTTGGGGTTG- |
| | ATATGATGTAAT-3′ | AAAAACCTCTTTAC-3′ | 3′ |
| cg23910243 | 5′- | 5′-Biotin- | 5′- |
| | AGGAGGTTGGGTTTGT | AACCAAAACCAAACAA | GGTTATAAATATTTTTT |
| | GT-3′ | AACACT-3′ | GTTATTTT-3′ |
| cg22187630 | 5′- | 5′-Biotin- | 5′- |
| | ATGGGGTTGTAGAGTG | AAACCCAAAAAATATA | GTTGTAGAGTGTTATG |
| | TTAT-3′ | CAAACAATCAAT-3′ | GT3′ |

Methylation Assays for MSRE qPCR Analysis

MSRE qPCR assays were designed for each DNAme marker using the Primer3Plus web tool (Untergasser A, et al. “Primer3Plus, an enhanced web interface to Primer3”. Nucleic Acids Res. 2007. The genomic region around the target was identified using the UCSC genome browser (http://genome.ucsc.edu/) and the sequence upstream and downstream of the predefined target region was extracted in FASTA file format. An identical number of base pairs was extracted on both sides of the target region and the final sequence adjusted to a length of approximately 300 bp. The FASTA file was imported into the Primer3 online tool and primers were designed by the software (see Table 2).

TABLE 2A
Example of oligonucleotide sequences,
theoretical Tm and modifications of MSRE
assay primers and probes.

| DNAme | Primer/ | Sequence | Tm | Modifi- |
|---|---|---|---|---|
| assay | probe | (5′-3′) | (°C) | cation |
| CG08202494 | CG08202494msre_F | CCCGTTTTGACTTTGCCAA | 58.2 | FAM-MGB |
| | CG08202494msre_R | CTCGAGAGGGCTTTCTGTCC | 57.7 | |
| | CG08202494msre_P | TCAATTTACTGAGGCCCGAG | 68.0 | |
| CG09010107 | CG09010107msre_F | TCCATCCATCAGCCTACCAC | 60.8 | FAM-MGB |
| | CG09010107msre_R | CAGGCACGTACAGACCTTTG | 60.2 | |
| | CG09010107msre_P | CGGTACCTGAGGGCCCTAG | 65.2 | |
| ACTB | ACTBmsp_F | AGGCCTGGACTCTCAACTGTG | 58.0 | VIC-MGB |
| | ACTBmsp_R | ACTGCAGAAATCAGACCAAAAGAG | 57.7 | |
| | ACTBmsp_P | TAGAACCACCCCAGAGAG | 68.0 | |

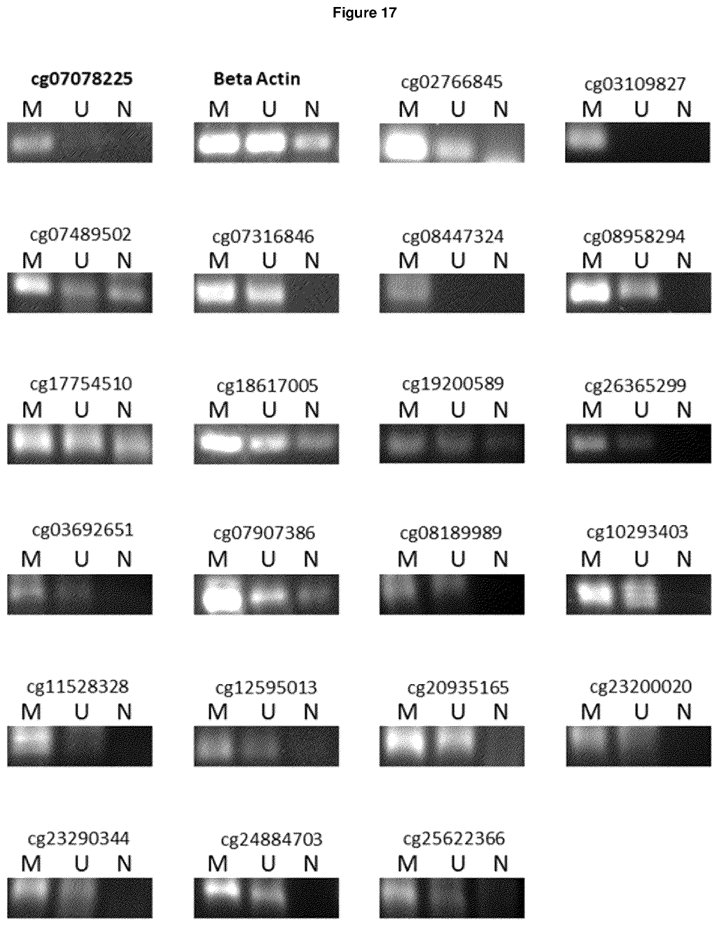

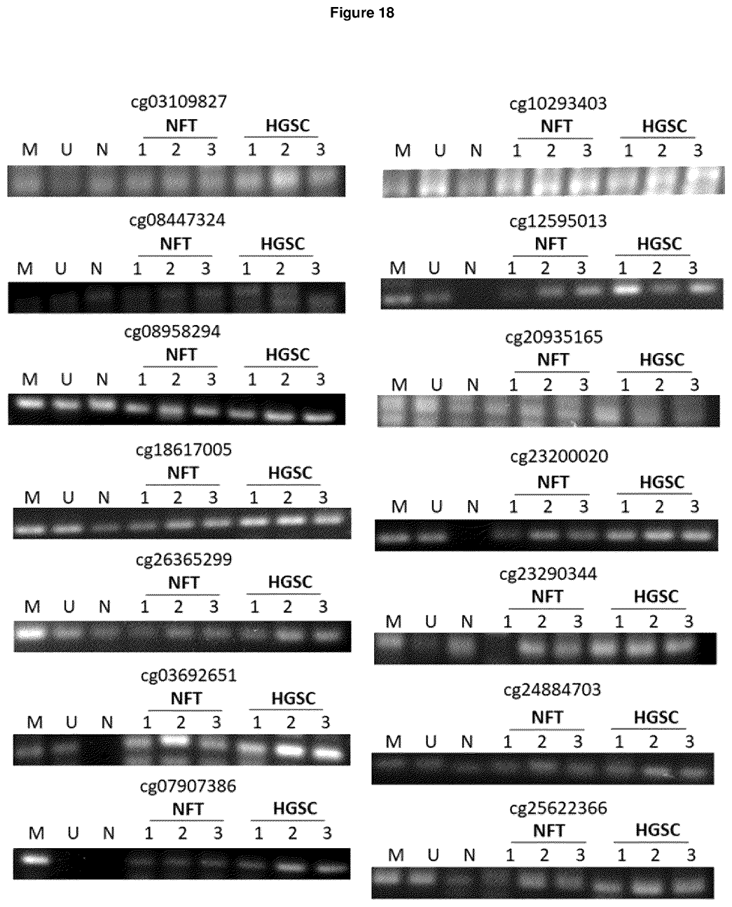

TABLE 2B
qPCR primers to be used for MSRE-DDPCR
(semi-quantitative PCRs using these
primer sets are shown in FIGS. 17 and 18)

| Target ID | Forward primer | Reverse primer |
|---|---|---|
| cg02766845 | AAGGGAAGGGCGTCTA | AACTTCAGGCAGCTGTT |
| | GGAT | CAG |
| cg03109827 | ACTCAAGGGCAAGCGG | CGAGGAGGCCAGGTAGA |
| | AA | GA |
| cg07489502 | AGTCTTGGATAGGGAA | CTCTGGGATTGTTCTCT |
| | CAGGT | GGGA |
| cg07316846 | ATGGGGTTCTGAAGGC | CACTGCTGCACTGTTCT |
| | TCC | TG |
| cg08447324 | CCTCCAGGGCTTGCTT | GGGCAGCCAGGAGAAGT |
| | ACT | C |
| cg08958294 | GGACCAGGAAACGACT | ACATCCTGATATCGGCT |
| | TAAGG | GCA |
| cg17754510 | CGTCCACATTTCCCTC | TTGATTTGGACTGTCTG |
| | CCT | GCG |
| cg18617005 | TCGGAGACCGAATTCA | GCTGGTTTTCTTCTTTC |
| | AAAT | TGTTG |
| cg19200589 | AAGCGGAGTTTGCCCT | ACTCCTTCCTTGACGCT |
| | GC | GC |
| cg26365299 | ATCACTCCGCACGCTA | TCTTTTAATCGTCGTTG |
| | TTAA | GCCA |
| cg03692651 | CCAGTGGCTTTCGTGG | CCCAGCTCATTCACTTG |
| | GC | TCA |
| cg07907386 | GTGTAGATGTCGGGGT | TCCTCTGTCATACTCTA |
| | AGTGA | GTTCCT |
| cg08189989 | CATCCTGTAGCGCCTC | CCGTTTCCCTTGCAGTC |
| | CTG | TAG |
| cg10293403 | CGTCTCCACCCTCAGT | AGCTCACCCTGTCCATT |
| | ACTT | CC |
| cg11528328 | TAAGCAGCGTCAAGGA | CTCTCCGGGTCTCTCAG |
| | AGGA | C |
| cg12595013 | GCAGCCTTCAACTCCA | CCGATGCAGCAAAGAGG |
| | CG | C |
| cg20935165 | GCAGCAGAGTCCGACC | TGCCCCTTCCAATCTGT |
| | TC | TCC |
| cg23200020 | CGGTAGGTATGCGGGT | CCCTACCCTCCCTGTCC |
| | CAC | A |
| cg23290344 | TCCCCGTCCAGTGGCT | TGAAGTCAAGGCTGCTC |
| | TC | TCG |
| cg24884703 | GGAGCGGGGTTGGATC | TCCACAAGTCTACCGTC |
| | AA | TGT |
| cg25622366 | TCGGCTTTCTTTTGCG | GAACGCGTGAAGGTGGT |
| | AAGG | G |

Template DNA was digested with two restriction enzymes: HpaII, and AciI. Both enzymes are CpG methylation sensitive and digestion only occurs if the internal cytosine is unmethylated. Genomic DNA was digested with methylation-sensitive restriction endonuclease HpaII and AciI. Reactions contained DNA (varying input amounts), 1× CutSmart® Buffer, 5-10 U HpaII (NEB, UK), 5-10 U AciI (NEB, UK) and nfH20 in a 50 μl total volume. Reactions were thoroughly mixed by pipetting up and down and incubated at 37° C. for at least 1 hour using an Eppendorf Mastercycler Gradient PCR Thermal Cycler. Digestion was stopped by heat inactivation at 80° C. for 20 minutes. DNA was either used immediately or stored at −20° C.

The products following MSRE digestion were quantified by real-time quantitative PCR using the same MSRE qPCR assays as mentioned above, specific for a region containing at least three enzyme digestion sites. DNA methylation status was calculated relative to ACTB, a reference control target with no restriction enzyme cut sites.

Sample Collection

All patient samples used were retrieved through registered tissue banks. The pilot cohort, which comprised six patients, was identified retrospectively from women who were diagnosed and treated at the Northern Ireland Gynaecological Cancer Centre (NIGCC), Belfast Health and Social Care Trust (BHSCT), Belfast. Formalin-fixed paraffin embedded (FFPE) tissue was retrieved from the BHSCT pathological archive. This was carried out under the scientific and ethical approval of the Northern Ireland Biobank (NIB) (NIB11:0005). The patients were selected based on the availability of NFT, STIC and HGSC within the resection specimens.

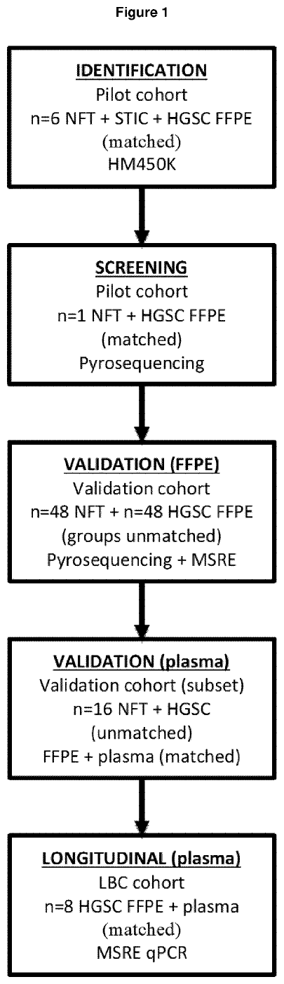

The validation cohort comprised two groups; non-cancerous and high-grade serous cancerous. The sample cohort was collated both retrospectively and prospectively. A subset of this cohort had matched FFPE tissue and plasma samples taken at the time of surgery. All patient samples were collected through the NIB with ethical approval obtained through the NIB Scientific Committee (NIB13:0094, NIB17:00235). FIG. 1 summarises the patient cohorts used for marker identification, screening and validation.

DNA Isolation

Newly extracted DNA from FFPE tissue was obtained through the NIB. Circulating DNA was extracted from 2 millilitres of plasma (due to limited amount availability) via a double centrifugation protocol of 1000 g for 10 min, followed by 16000 g for 10 min. cfDNA was extracted from 2 ml of blood plasma using the QIAamp® Circulating Nucleic Acid Kit (Qiagen, Manchester, UK). All samples were stored at −80° C. prior to further processing.

Statistics and Data Analyses

Statistical analyses were performed using the GraphPad Prism 8 software (La Jolla, Calif., USA). The software was used to calculate p values which are presented as (*) in the results. * denotes a p value <0.05, ** denotes a p value <0.01 and *** a p value <0.001. The particular statistical tests used are indicated in the brief description of the drawings.

Receiver-operating characteristic (ROC) analysis was carried out and area under the curve (AUC) was calculated to evaluate the diagnostic performance of DNAme markers. p<0.05 was considered to indicate a statistically significant difference.

### Example 1

Methylation Marker Discovery

Previous work in Beirne JP. “The identification and characterisation of disease-specific biomarkers in pelvic high grade serous carcinomas” 2016 identified candidate DMRs, whereby DNAme profiling was carried out using the Illumina® Infinium Human Methylation 450K BeadChip® platform on the same pilot cohort analysed herein. An analysis of differential methylation was conducted according to the following comparisons: (1) NFT-HGSC, (2) NFT-STIC and (3) STIC-HGSC.

β values reported by the 450K Illumina® platform for each probe represent the methylation level measurement for the targeted CpG site. The range of the β value is from 0 (no methylation) to 1 (100% methylation). A higher β value indicates a higher DNAme level. Differential methylation was computed based on the difference in mean β values (methylation levels) of the two groups being compared. The candidate DMRs for this study were identified based on the top ranking differentially hypermethylated CpG sites within the NFT-HGSC comparison, as determined in the previous study.

TABLE 3A
Summary of top ranking 20 DMRs within the NFT-HGSC comparison.

| | | | Relation | |
|---|---|---|---|---|
| | Associated Gene; | | to CpG | |
| DMRs | Accession No; Gene ID | Gene type | island | Chromosome |
| cg08202494* | OSR2; NM_001142462; | Transcription | Shore | 8 |
| | 116039 | factor | | |
| cg01657761 | LINCO1197; NR_034095.1; | Non-protein | Data not | 2 |
| | 400456 | coding RNA | available | |
| cg03035213 | ZNF469; NM_032752.3; | Transcription | Island | 16 |
| | 84838 | factor | | |
| cg03314029 | ZNF469; NM_032752.3; | Transcription | Island | 16 |
| | 84838 | factor | | |
| cg04453471_A | MAP3K8; NM_005204.4; | Oncogene | Island | 10 |
| | 1326 | | | |
| cg04453471_B | MAP3K8; NM_005204.4; | Oncogene | Island | 10 |
| | 1326 | | | |
| cg07215504 | ZNF469; NM_032752.3; | Transcription | Island | 16 |
| | 84838 | factor | | |
| cg11469908 | LINC01798; NR_110156.1; | Non-protein | Data not | 2 |
| | 100507073 | coding RNA | available | |
| cg15712559 | PHACTR3; NM_080672.5; | Protein coding | Island | 20 |
| | 116154 | gene | | |
| cg16329896 | TNS3; NM_022748.12; 64759 | Protein coding | Data not | 7 |
| | | gene | available | |
| cg05224741 | TFAP2A/LINC00518; | Transcription | Island | 6 |
| | NM_001372066.1; 7020/ | factor/Non-protein | | |
| | NR_027793.1; 221718 | coding RNA | | |
| cg09010107 | PRRX1; NM_006902.5; 5396 | Transcription co- | Data not | 1 |
| | | activator | available | |
| cg04043571 | NR5A1; NM_004959.5; 2516 | Protein coding | Island | 9 |
| | | gene | | |
| cg08610862 | LHX9; NM_020204.3; 56956 | Transcription | Shore | 1 |
| | | factor | | |
| cg13912311 | NR5A1; NM_004959.5; 2516 | Protein coding | Island | 9 |
| | | gene | | |
| cg23044884 | RBPMS; NM_001008710.3; | Protein coding | Shelf | 8 |
| | 11030 | gene | | |
| cg14284618 | TACC1; NM_006283.3; 6867 | Protein coding | Data not | 8 |
| | | gene | available | |
| cg15511120 | DNHD1; NM_144666.3; | Protein coding | Data not | 11 |
| | 144132 | gene | available | |
| cg23910243 | TGFB1I1; NM_001042454.3; | Protein coding | Shore | 16 |
| | 7041 | gene | | |
| cg22187630 | CACNA1A; NM_000068.4; | Protein coding | Island | 19 |
| | 773 | gene | | |
| cg07078225 | KRT87P; NR_146088.1; | Pseudogene | Island | 12 |
| | 85349 | (ncRNA) | | |

TABLE 3B
Summary of top ranking 21 DMRs within the NFT-HGSC comparison.

| | Associated Gene; | | Relation to | |
|---|---|---|---|---|
| DMRs | Accession No; Gene ID | Gene type | CpG Island | Chromosome |
| cg02766845 | HCG15; NR_145490; 414761 | long non-coding | Island | 6 |
| | | RNA | | |
| cg03109827 | TCERG1L; NM_174937; | Protein coding | Island | 10 |
| | 256536 | gene | | |
| cg07489502 | PCDHGA4; NM_018917; | Protein coding | Island | 5 |
| | 56111 | gene | | |
| cg07316846 | LINC01159; NR_110373; | long non-coding | S_Shore | 2 |
| | 102682016 | RNA | | |
| cg08447324 | SHANK1; NM_016148; | Protein coding | Island | 19 |
| | 50944 | gene | | |
| cg08958294 | GRM1; NM_001278064-7; | Protein coding | N_Shore | 6 |
| | 2911 | gene | | |
| cg17754510 | TFAP2A; NM_001372066.1; | Transcription | S_Shore | 6 |
| | 7020 | factor | | |
| cg18617005 | PCDHGA4; NM_018917; | Protein coding | Island | 5 |
| | 56111 | gene | | |
| cg19200589 | CLIC6; NM_001317009; | Protein coding | Island | 21 |
| | 54102 | gene | | |
| cg26365299 | HOXA9; NM_152739; 3205 | Transcription | Island | 7 |
| | | factor | | |
| cg03692651 | ZNF729; NM_001242680; | Transcription | Island | 19 |
| | 100287226 | factor | | |
| cg07907386 | PHOX2B; NM_003924; 8929 | Transcription | Island | 4 |
| | | factor | | |
| cg08189989 | PANTR1/LINC01158; | long non-coding | Island | 2 |
| | NR_037883; 100506421 | RNA | | |
| cg10293403 | TFAP2A; NM_001372066.1; | Transcription | Island | 6 |
| | 7020 | factor | | |
| cg11528328 | CLIC6; NM_001317009; 54102 | Protein coding | Island | 21 |
| | | gene | | |
| cg12595013 | ZIC1; NM_003412; 7545 | Transcription | Island | 3 |
| | | factor | | |
| cg20935165 | DLX1/DLX-DT; NM_178120; | Transcription | Island | 2 |
| | 1745 | factor | | |
| cg23200020 | SCGB1B2P; NR_027620; | Pseudogene | Island | 19 |
| | 643719 | | | |
| cg23290344 | NEFM; NM_005382; 4741 | Protein coding | Island | 8 |
| | | gene | | |
| cg24884703 | BARHL2; NM_020063; 343472 | Transcription | Island | 1 |
| | | factor | | |
| cg25622366 | OTX1; M_014562; 5013 | Transcription | Island | 2 |
| | | factor | | |

Any reference to any genomic coordinates is made with reference to Genome Reference Consortium Human Build 37 (GRCh37) for Homo sapiens (human) as part of the BioProject: PRJNA31257 submitted by Genome Reference Consortium on 27 Feb. 2009 having GenBank assembly accession: GCA_000001405.1 and RefSeq assembly accession: GCF_000001405.13.

TABLE 4
Summary of the mean methylation levels for the candidate DMRs.

| | Mean | Mean | Mean | Stdev | Stdev | Stdev |
|---|---|---|---|---|---|---|
| Target ID | NFT | STIC | HGS | NFT | STIC | HGS |
| cg08202494 | 0.385174 | 0.514348 | 0.822006 | 0.025232 | 0.120252 | 0.046811 |
| cg03035213 | 0.123876 | 0.197632 | 0.546648 | 0.055547 | 0.216787 | 0.163849 |
| cg03314029 | 0.201166 | 0.207942 | 0.667472 | 0.051306 | 0.200347 | 0.104398 |
| cg04043571 | 0.209964 | 0.256438 | 0.732842 | 0.045606 | 0.080264 | 0.09915 |
| cg04453471 | 0.204479 | 0.311722 | 0.576884 | 0.019489 | 0.110779 | 0.110909 |
| cg07215504 | 0.111949 | 0.157454 | 0.519475 | 0.011501 | 0.153994 | 0.165461 |
| cg11469908 | 0.294852 | 0.481908 | 0.72931 | 0.025199 | 0.14883 | 0.10565 |
| cg15712559 | 0.173558 | 0.169532 | 0.597592 | 0.032879 | 0.045672 | 0.180931 |
| cg16329896 | 0.202948 | 0.554523 | 0.731403 | 0.051062 | 0.11044 | 0.013575 |
| cg05224741 | 0.063121 | 0.483642 | 0.530689 | 0.01408 | 0.262766 | 0.274664 |
| cg09010107 | 0.400109 | 0.53117 | 0.793343 | 0.077289 | 0.118413 | 0.08417 |
| cg24376434 | 0.450999 | 0.623606 | 0.84263 | 0.044718 | 0.082171 | 0.029509 |
| cg08610862 | 0.31456 | 0.470804 | 0.659828 | 0.035248 | 0.123659 | 0.14076 |
| cg13912311 | 0.252483 | 0.402461 | 0.825549 | 0.056808 | 0.085879 | 0.135783 |
| cg23044884 | 0.376262 | 0.543627 | 0.720328 | 0.038685 | 0.062343 | 0.11539 |
| cg20343048 | 0.391481 | 0.528469 | 0.759161 | 0.063433 | 0.129403 | 0.113404 |
| cg15511120 | 0.364378 | 0.477298 | 0.673552 | 0.039964 | 0.064096 | 0.0987 |
| cg23910243 | 0.462012 | 0.622451 | 0.765299 | 0.030717 | 0.08879 | 0.081969 |
| cg14284618 | 0.332951 | 0.459462 | 0.734885 | 0.080025 | 0.124266 | 0.114039 |
| cg22187630 | 0.188803 | 0.255257 | 0.397245 | 0.048195 | 0.129914 | 0.199608 |
| cg07078225 | 0.247679 | 0.57387 | 0.629629 | 0.068713 | 0.115649 | 0.194275 |
| cg02766845 | 0.121158 | 0.389874 | 0.518493 | 0.037955 | 0.138955 | 0.345787 |
| cg03109827 | 0.120458 | 0.363061 | 0.551092 | 0.054102 | 0.106098 | 0.266658 |
| cg07489502 | 0.184072 | 0.516942 | 0.615203 | 0.064160 | 0.177333 | 0.266552 |
| cg07316846 | 0.121141 | 0.482637 | 0.498733 | 0.031632 | 0.213084 | 0.350158 |
| cg08447324 | 0.091869 | 0.310938 | 0.514284 | 0.019158 | 0.217412 | 0.335348 |
| cg08958294 | 0.100832 | 0.384312 | 0.53545 | 0.010541 | 0.184912 | 0.297020 |
| cg17754510 | 0.128514 | 0.597103 | 0.643632 | 0.109014 | 0.328999 | 0.238144 |
| cg18617005 | 0.116848 | 0.472543 | 0.495812 | 0.018664 | 0.186427 | 0.351642 |
| cg19200589 | 0.144012 | 0.412058 | 0.69615 | 0.147820 | 0.236336 | 0.196805 |
| cg26365299 | 0.148275 | 0.383114 | 0.497522 | 0.025101 | 0.122841 | 0.345275 |
| cg03692651 | 0.170091 | 0.466862 | 0.545163 | 0.043580 | 0.184125 | 0.300078 |
| cg07907386 | 0.12092 | 0.349862 | 0.541081 | 0.048952 | 0.150349 | 0.290458 |
| cg08189989 | 0.112938 | 0.375337 | 0.494105 | 0.068136 | 0.257740 | 0.379065 |
| cg10293403 | 0.139978 | 0.42631 | 0.536451 | 0.032629 | 0.176960 | 0.241676 |
| cg11528328 | 0.264297 | 0.508228 | 0.667161 | 0.103738 | 0.212749 | 0.140830 |
| cg12595013 | 0.147988 | 0.473574 | 0.549544 | 0.045148 | 0.145852 | 0.285651 |
| cg20935165 | 0.111988 | 0.447026 | 0.496681 | 0.026365 | 0.208084 | 0.282896 |
| cg23200020 | 0.119792 | 0.454864 | 0.492101 | 0.055490 | 0.214290 | 0.416271 |
| cg23290344 | 0.144605 | 0.491598 | 0.572983 | 0.048424 | 0.202030 | 0.294975 |
| cg24884703 | 0.155931 | 0.475337 | 0.524168 | 0.040126 | 0.221978 | 0.333112 |
| cg25622366 | 0.151506 | 0.546122 | 0.582007 | 0.060275 | 0.194351 | 0.298452 |

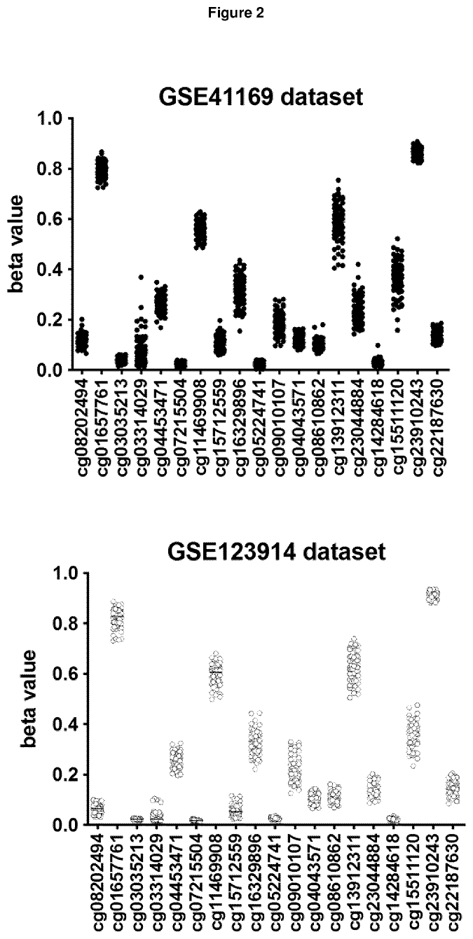

In-silico analysis using the GEO datasets was performed on the 21 candidate DMRs. FIG. 2 shows the β values for 20 of the candidate DMRs according to the GSE41169 and GSE123914 datasets. DMRs with β values <0.2 were selected for further evaluation.

### Example 2

Validation of Methylation Markers in Tissue Using Pyrosequencing

Given constraints on resources, specifically the availability of DNA from FFPE tissue samples from the pilot study cohort, a limited number of nine DNAme markers (β values <0.2) were chosen for further evaluation in tissue (see Table 5).

TABLE 5
DNAme markers (β values < 0.2) chosen for further evaluation in tissue.
DNAme marker

cg08202494*
cg05224741
cg13912311
cg15712559 v1
cg09010107
cg04453471_B v2
cg04453471_A
cg11469908
cg23910243

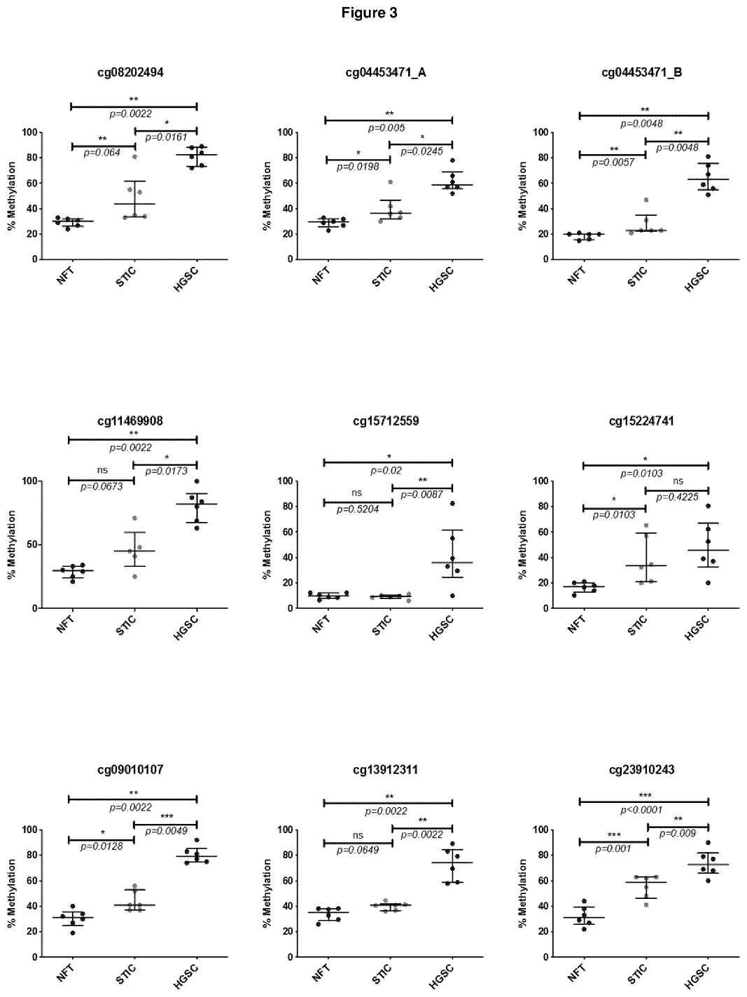

Assays were analysed using matched NFT, STIC and HGSC FFPE tissue taken from the original pilot study cohort. FIG. 3 shows scatter plots comparing NFT, STIC and HGSC median methylation scores following pyrosequencing for each DNAme marker. Methylation scores were significantly increased in the HGSC group compared to the NFT group for all DNAme markers as analysed by Kruskal-Wallis H test (see FIG. 3 and Table 6). Further inter-group differences (NFT-STIC, STIC-HGSC, NFT-HGSC) were assessed by Mann-Whitney U test, and significant differences were found in all DNAme markers comparing NFT-HGSC (see FIG. 3). There was no statistically significant difference found between NFT-STIC in three of the nine DNAme markers (cg11469908, p=0.5204; cg15712559, p=0.0673; cg13912311, p=0.0649).

TABLE 6
Inter-group analysis of NFT/STIC/HGSC for top nine performing DNA methylation markers

| | NFT | | | STIC | | | HGSC | | | K-W |
|---|---|---|---|---|---|---|---|---|---|---|
| | | | 95% | | | 95% | | | 95% | analysis |
| DMR | Median | IQR | CI | Median | IQR | CI | Median | IQR | CI | Pvalue |
| cg08202494 | 30.00 | 26.65- | 25.78- | 44.00 | 33.75- | 28.86- | 82.50 | 73.50- | 73.89- | 0.0009 |
| | | 32.35 | 32.89 | | 61.50 | 68.14 | | 88.25 | 88.77 | * |
| cg09010107 | 31.00 | 25.00- | 22.92- | 41.00 | 37.00- | 35.55- | 79.00 | 74.75- | 73.32- | 0.0007 |
| | | 35.50 | 37.74 | | 53.00 | 52.45 | | 85.25 | 87.35 | * |
| cg13912311 | 35.15 | 28.60- | 28.25- | 40.70 | 36.60- | 36.70- | 74.45 | 58.65- | 59.39- | 0.0015 |
| | | 38.15 | 39.12 | | 42.06 | 43.28 | | 84.60 | 86.58 | * |
| cg23910243 | 31.00 | 25.75- | 23.84- | 58.50 | 46.25- | 45.70- | 73.00 | 66.00- | 62.86- | 0.0011 |
| | | 39.50 | 40.49 | | 63.00 | 64.97 | | 81.75 | 84.81 | * |
| cg04453471_A | 29.50 | 26.00- | 25.19- | 36.50 | 32.25- | 28.16- | 59.00 | 55.75- | 52.18- | 0.0018 |
| | | 32.35 | 32.81 | | 46.74 | 51.51 | | 69.00 | 71.48 | * |
| cg04453471_B | 20.00 | 15.75- | 16.04- | 23.00 | 22.50- | 17.57- | 63.00 | 54.75- | 52.67- | 0.0005 |
| | | 20.25 | 21.29 | | 35.00 | 38.43 | | 75.75 | 76.66 | * |
| cg11469908 | 29.50 | 24.00- | 23.50- | 45.00 | 33.00- | 25.45- | 82.00 | 67.50- | 66.63- | 0.0029 |
| | | 33.25 | 33.84 | | 59.50 | 66.55 | | 90.25 | 94.37 | * |
| cg15712559 | 9.75 | 8.00- | 7.45- | 9.25 | 7.87- | 7.18- | 36.13 | 24.63- | 15.51- | 0.0126 |
| | | 12.25 | 12.21 | | 10.50 | 10.99 | | 61.88 | 67.57 | * |
| cg15224741 | 17.30 | 13.08- | 12.47- | 33.45 | 20.98- | 18.77- | 45.80 | 32.75- | 26.21- | 0.0069 |
| | | 20.25 | 20.83 | | 59.08 | 58.06 | | 66.88 | 70.96 | * |

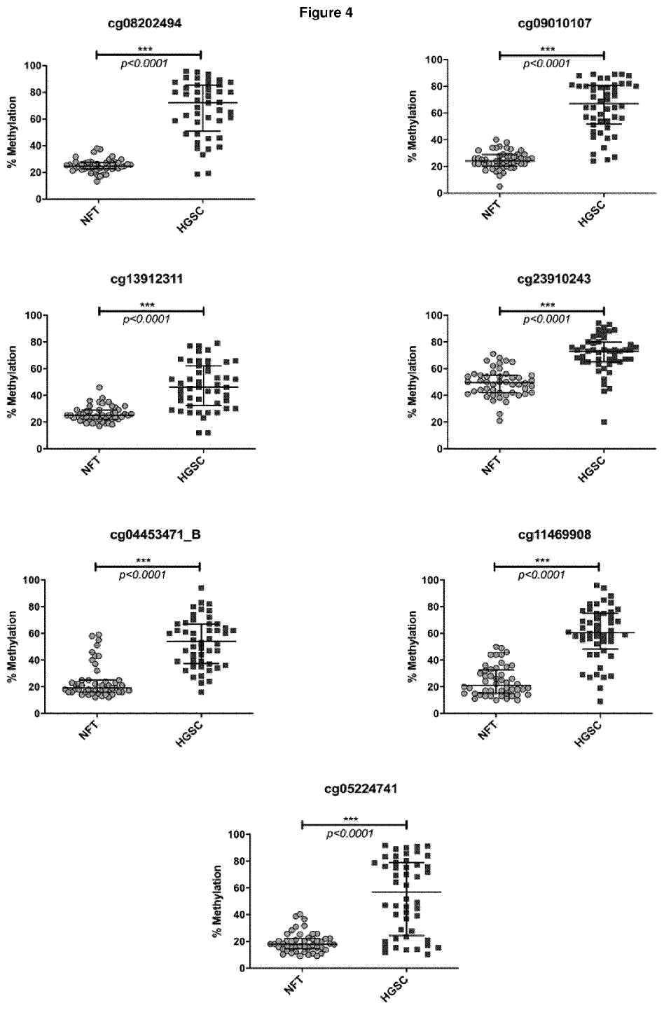

Of the nine candidate DNAme markers validated in the pilot study cohort, seven were taken forward for further evaluation in the validation cohort of 48 non-cancerous NFT and 48 HGSC FFPE tissue samples (see FIG. 4).

At this stage, cg04453471_A and cg15712559 were excluded for the following reasons: cg04453471_A and cg04453471_B represent alternative pyrosequencing assays for the same region of interest. cg04453471_B showed improved statistical performance compared to cg04453471_A following analysis in the pilot cohort and was therefore selected to take forward for further evaluation in the validation cohort. cg15712559 was excluded as the median methylation score was lower in STIC compared to NFT for this marker (NFT 9.75%, STIC 9.25%). cg11469908 and cg13912311 did not show statistically significant hypermethylation from NFT-STIC (p=0.0673 and p=0.0649, respectively). However, there was an apparent trend towards increased methylation in the STIC group (median methylation scores increasing from 29.50% to 45.00% for cg11469908 and 35.15% to 40.70% for cg13912311) and a decision was made to include these DNAme markers in those taken forward for further evaluation in the validation cohort.

### Example 3

ROC Analyses of Methylation Markers Compared to CA125

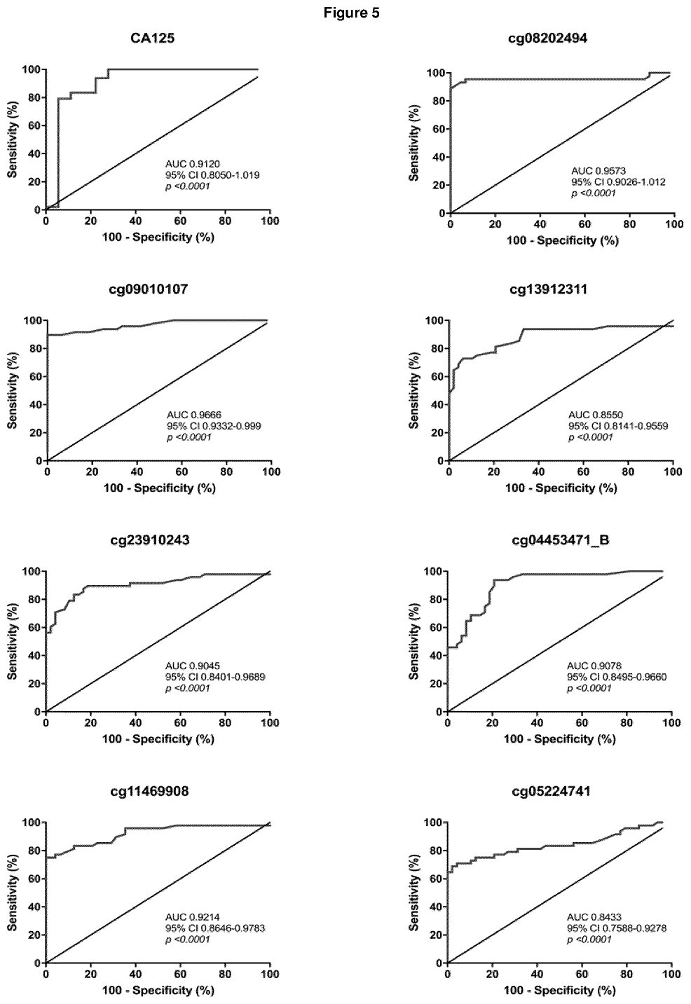

Receiver operating characteristic (ROC) analysis was performed to determine the diagnostic accuracy of the seven DNAme markers in detecting HGSC. FIG. 5 shows the ROC curves for CA125 and the seven DNAme markers following pyrosequencing analysis in the validation cohort. Youden's index was calculated for each DNAme marker to determine the maximum optimum cut-off threshold. This was calculated using the formula below and used to determine the corresponding sensitivity, specificity and cut-off value:

J=sensitivity+specificity−1

Having determined the sensitivity and specificity for each DNAme marker, these values were compared with the current gold standard, CA125. Specificity of all seven markers was higher than CA125. However, CA125 achieved a higher sensitivity compared to the DNAme markers. ROC analysis revealed three markers with improved overall diagnostic accuracy compared to CA125 (AUC, 0.912): cg08202494 (AUC, 0.9573), cg09010107 (AUC, 0.9666) and cg11469908 (AUC, 0.9214) (see FIG. 5).

### Example 4

Evaluation in Early Stage Disease

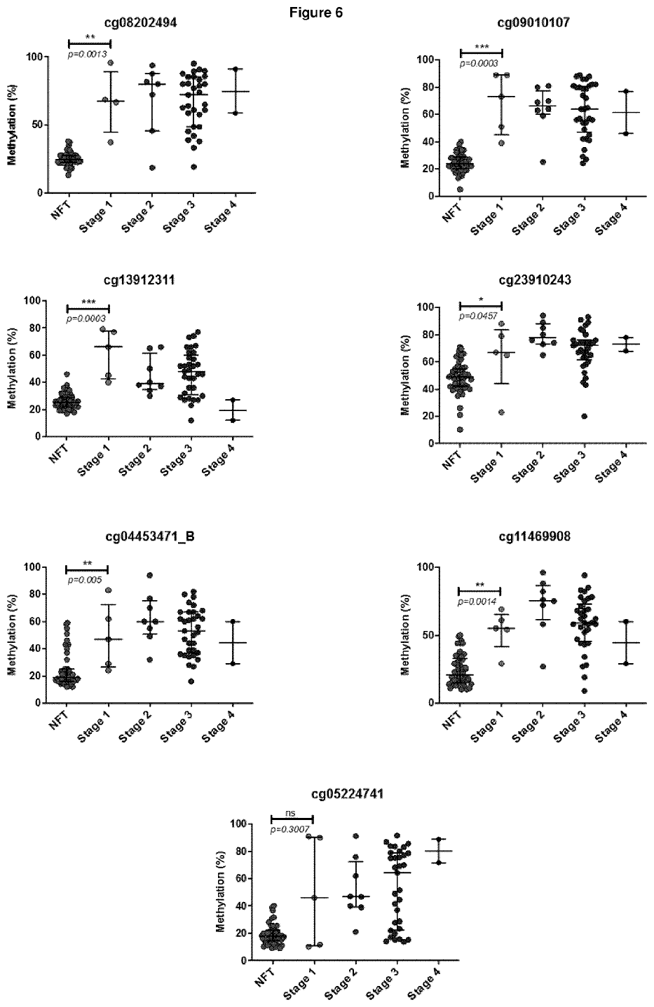

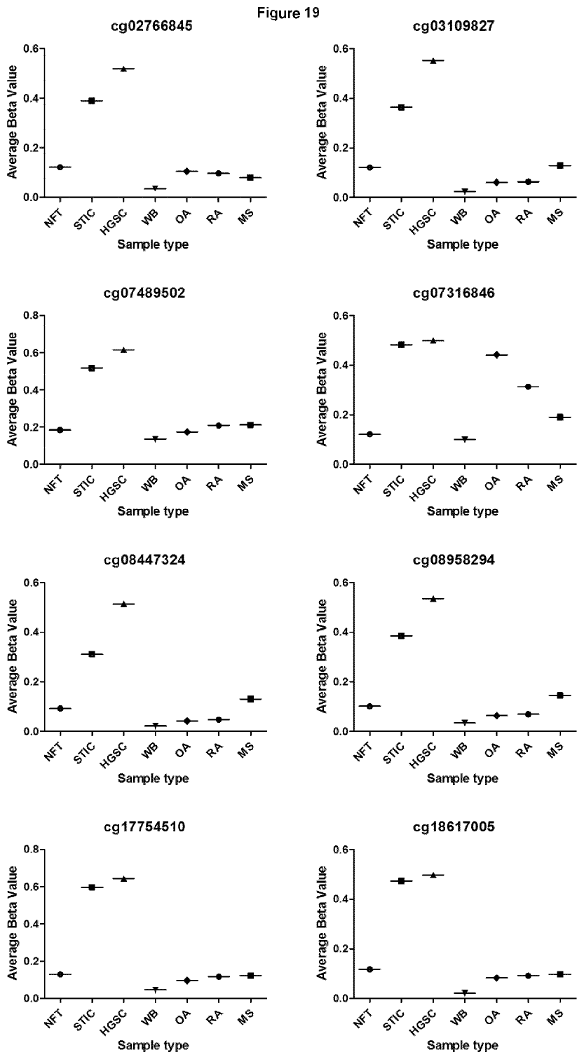

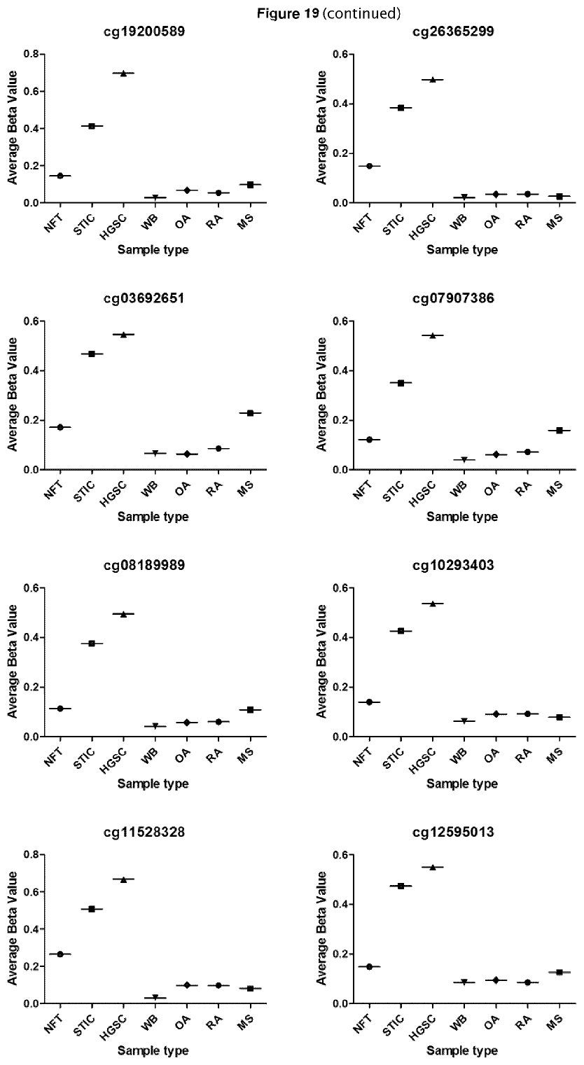

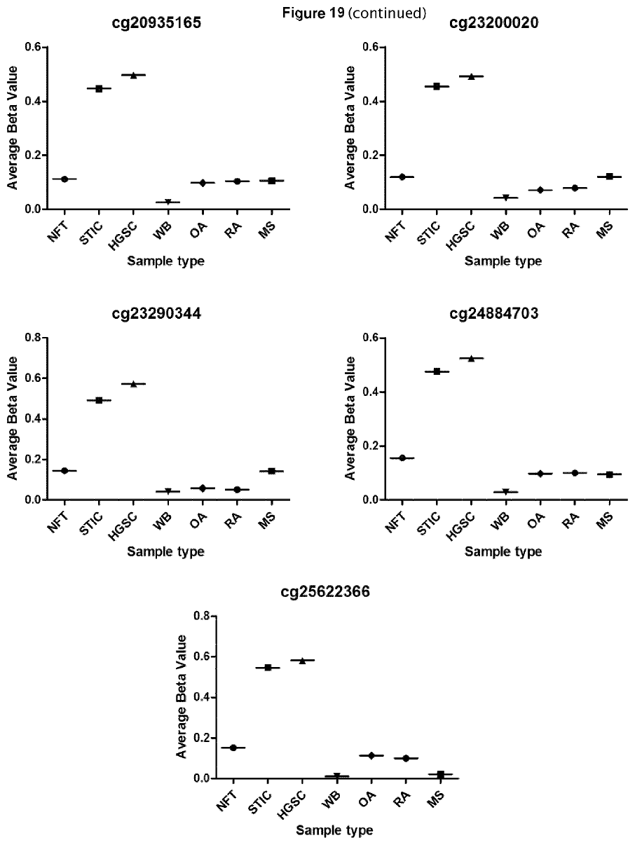

To further evaluate the potential for detection in early disease, methylation scores for the DNAme markers were stratified according to FIGO stage (see FIGS. 6 and 19).

Statistically significant hypermethylation was observed in FIGO stage 1 compared to NFT for six out of seven of the DNAme markers highlighting their potential in early detection. cg05224741 did not show statistically significant hypermethylation in FIGO stage 1 compared to NFT; however, this is likely due to the wide range of methylation scores observed in the FIGO stage 1 group (range, 10.30-91.00).

### Example 5

Validation of MSRE qPCR Assays in Tissue

Two DNAme markers, cg08202494 and cg09010107, showed superior diagnostic accuracy compared in CA125 when analysed in FFPE tissue samples using pyrosequencing. As the most promising candidates, these two markers were taken forward for further evaluation using MSRE qPCR.

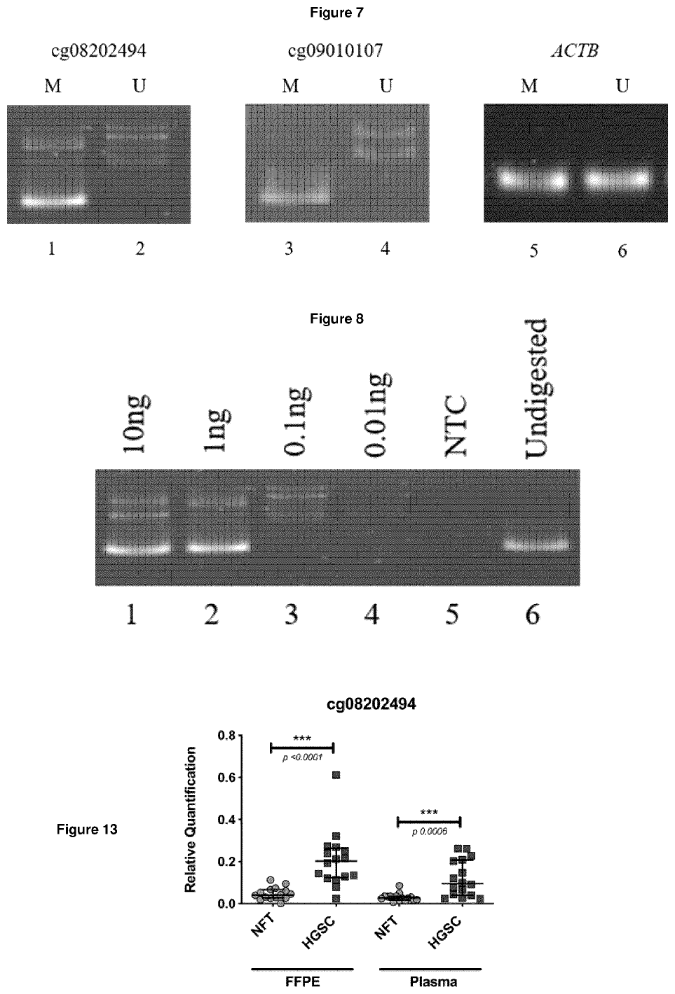

FIG. 7 shows that all assays, cg08202494, cg09010107 and ACTB, display appropriate specificity following restriction enzyme digestion in methylated and unmethylated controls. In the target assays, cg08202494 and cg09010107, the methylated control results in PCR product when digested with HpaII and AciI as the methylated cytosines are resistant to cleavage. In contrast, there is no product following digestion with these enzymes in the unmethylated control as the unmethylated cytosines within the amplicon sequence are cleaved, preventing amplification. In the ACTB reference control assay, product is seen in both the methylated and unmethylated controls as there are no cut sites for these enzymes within the ACTB amplicon. See also FIGS. 17 and 18.

To establish the lower limit of DNA input, digestion was carried out on Human genomic DNA (Roche, Germany) using input concentrations of 100 ng, 10 ng, 1 ng and 0.1 ng. One-tenth of the digestion mixture was used for PCR, so the concentration of the DNA template was 10 ng, 1 ng, 0.1 ng and 0.01 ng (see FIG. 8). A starting input of 10 ng was selected as the limit of detection. This is in keeping with previously reported DNA input limits for MSRE qPCR (range 10-100 ng).

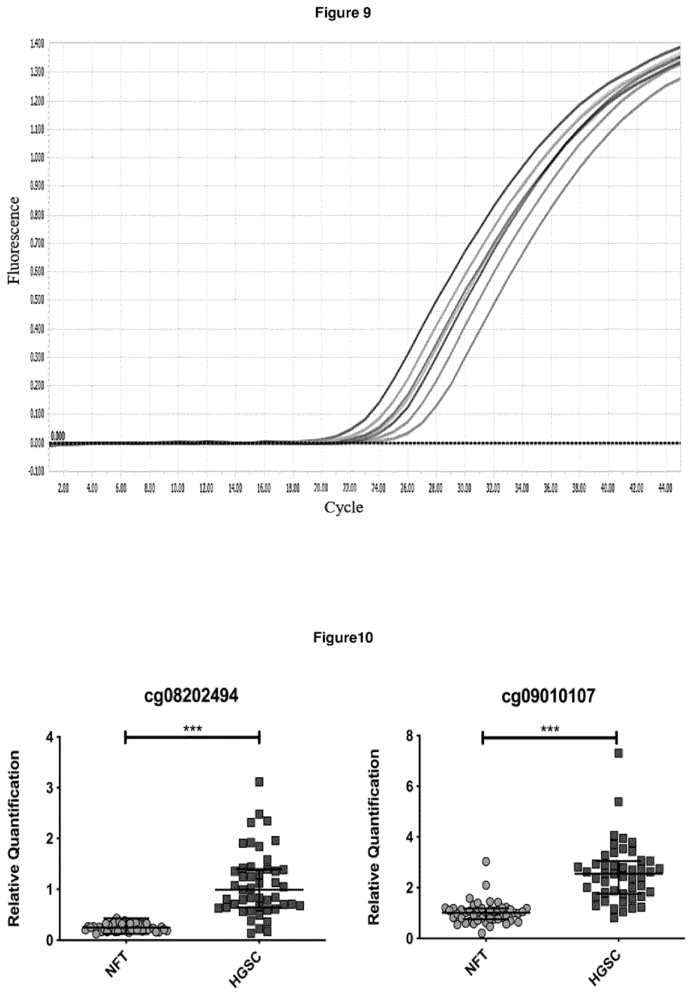

A calibration standard curve was prepared using serial dilutions of commercially available methylated and unmethylated DNA (Zymo Research, USA) using 10 ng input DNA. The calibration curve covered a methylation range of 100%, 80%, 40%, 20%, 10%, 5%, 2.5% (see FIG. 9).

Calibration standards were prepared and then digested with the restriction enzymes HpaII and AciI. The calibration standard curve, as well as positive and negative controls, was included in every qPCR run.

FIG. 10 shows the relative quantification (RQ) of cg08202494 and cg09010107 compared to the reference control ACTB in the validation cohort (48 NFT and 48 HGSC tissue samples). The RQ of both markers was statistically significantly increased in the HGSC cohort (p<0.0001).

### Example 6

Diagnostic Accuracy of MSRE qPCR Assays in Tissue

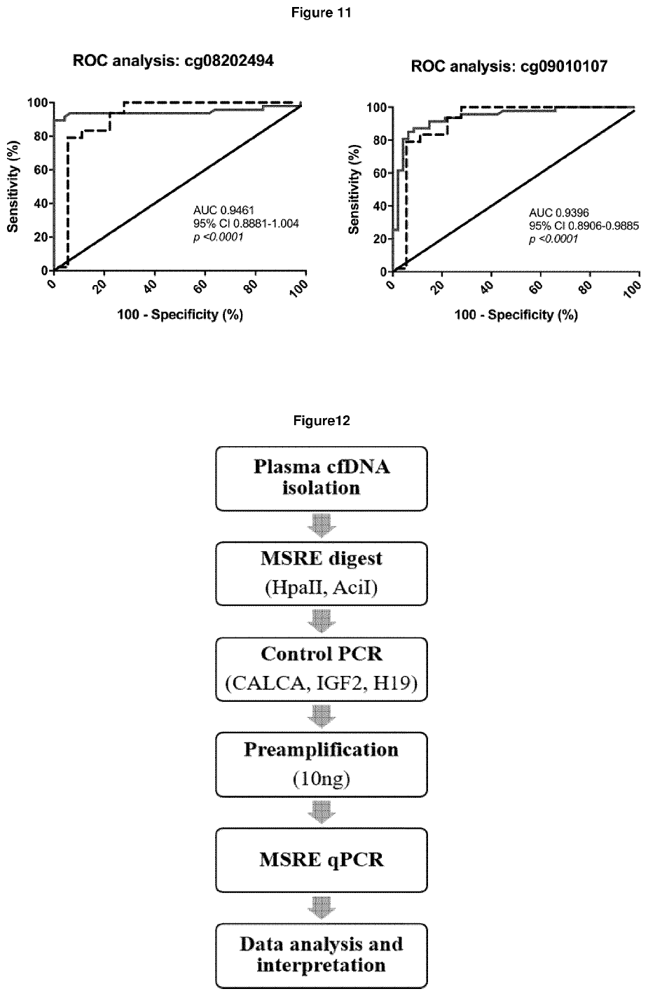

The receiver operating characteristics (ROC) curves were performed to evaluate the performance of cg08202494 and cg09010107 as biomarkers in distinguishing HGSC from NFT tissue samples. Both markers again showed improved diagnostic accuracy compared to CA125 (see FIG. 11).

### Example 7

Detection of Methylation Markers in Circulating Tumour DNA of Healthy Individuals and Ovarian Cancer Patients

The workflow adopted to optimise the MSRE qPCR assay for use on cfDNA is depicted in FIG. 12.

The assay was optimised using cfDNA extracted from ascitic fluid to mimic a starting material more comparable to plasma cfDNA than genomic DNA extracted from FFPE. Having optimised the MSRE assay workflow, the assay was then tested on a subset of 32 patients from the validation cohort (16 NFT and 16 HGSC) with matched FFPE tissue and plasma samples taken from the time of cytoreductive surgery. FIG. 13 shows the RQ of tissue samples compared to plasma samples in each group.

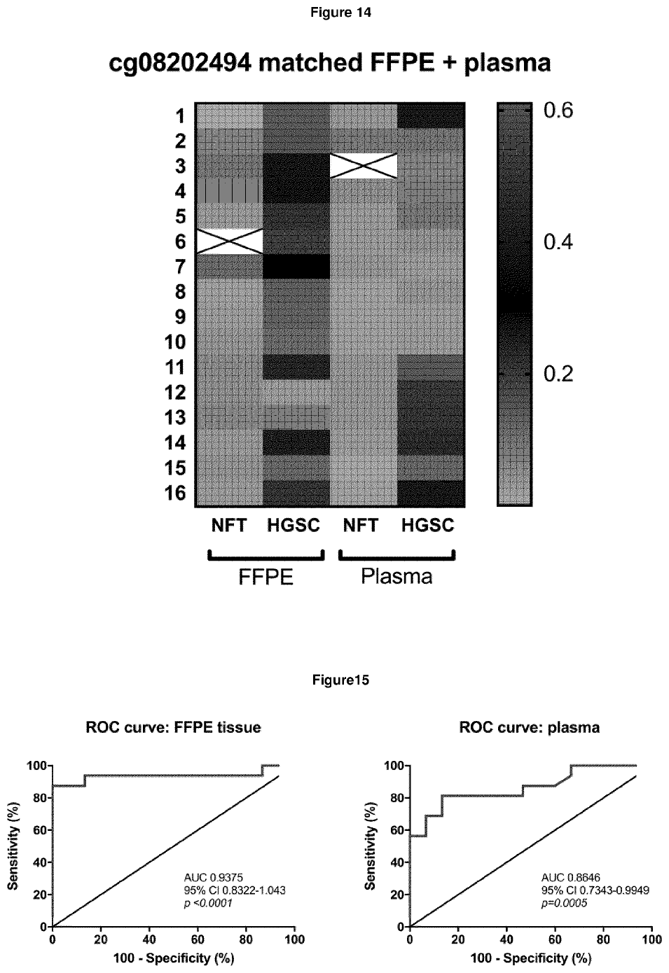

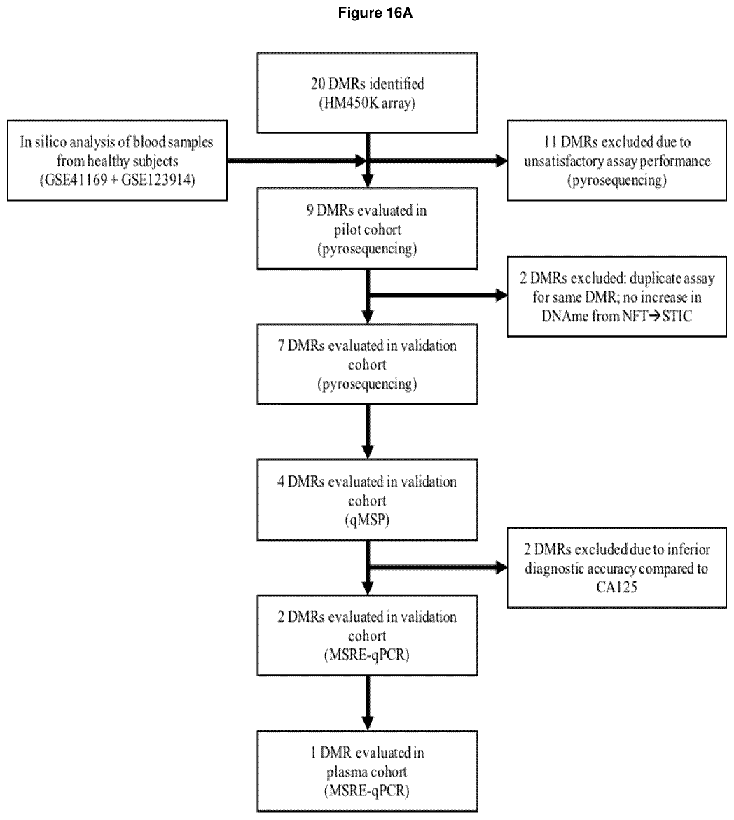

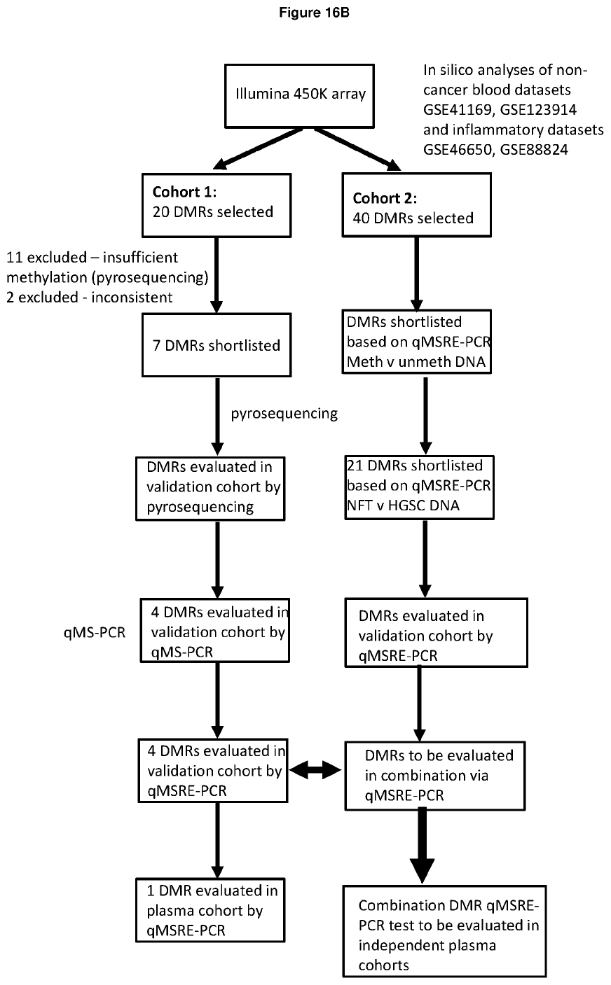

Statistically significant hypermethylation was observed in the HGSC group compared to the NFT group in both FFPE (p<0.001) and plasma (p=0.0006). FIG. 14 shows the correlation between individually matched FFPE and plasma samples for cg08202494 in this patient cohort showing a trend towards increased DNA methylation in the HGSC group.

Diagnostic accuracy was then assessed as previously described using ROC analysis (see FIG. 15).

AUC in the matched plasma samples (AUC 0.8646, p=0.0005) was lower than the corresponding tissue samples (AUC 0.9375, p<0.0001). The diagnostic accuracy observed in this plasma sample set was also lower compared to CA125 (AUC 0.912, p<0.0001).

The clinical need for the early detection of OC is indisputable. Early diagnosis with successful optimal cytoreductive surgery leads to significantly improved outcomes. Large scale prospective trials have shown that the current gold standard detection methods, CA125 and TVUS, cannot be recommended for screening purposes. Strikingly, despite advances in biomedical science and analytical technologies, no novel biomarker has been approved for screening or diagnosis in OC in the last decade.

Cancer is driven by progressive genetic alterations, such as mutations involving oncogenes and tumour suppressor genes. More recently, it has been demonstrated that cancer is also driven by epigenetic alterations. Epigenetic mechanisms are defined as heritable changes in gene expression that do not alter the primary DNA sequence. Epigenetic alterations can influence the transcriptional process, leading to changes in the expression of genes involved in cellular processes such as proliferation, differentiation and survival. The most commonly occurring epigenetic changes; DNAme, histone modification and nucleosomal remodelling, mutually interact to regulate gene expression.

DNAme, the best-known epigenetic mechanism, occurs predominantly on cytosines that precede a guanosine in the DNA sequence; a CpG dinucleotide. Clusters of these dinucleotides, termed CpG islands (CGI), are often associated with promoter regions of a gene. CGIs found in gene promoter regions are usually unmethylated in normal cells and this is understood to facilitate active gene transcription. In contrast, CpG sites outside the CGIs tend to be methylated and play a role in global genome stability.

DNAme patterns in cancer cells are significantly altered compared to those of normal cells and these alterations are thought to represent some of the earliest events in carcinogenesis. Changes in 5-methylcytosine distribution in cancer cells results in two predominant aberrant methylation patterns: (1) hypermethylation of CGIs, and (2) global DNA hypomethylation. Localised hypermethylation of CGIs in gene promoter regions and other regulatory regions, is a frequent mechanism of tumour suppressor gene (TSG) inactivation. Initiation of this process has been connected to increased levels of DNMTs. In contrast, global hypomethylation has been hypothesized to contribute to cancer development by transcriptional activation of oncogenes and chromosomal instability. Hypermethylation patterns are histopathological type specific, whereas hypomethylation patterns appear to be a ubiquitous feature of all cancers.

Surprisingly, despite minimal blood-borne spread, aberrant DNAme can be detected in serum, plasma and peritoneal fluid of OC patients. DNAme has several advantages compared to other molecular biomarkers. Methylation analysis utilises DNA which is chemically more stable than other molecules, such as RNA and protein. DNAme patterns are also chemically and biologically stable and are relatively unaffected by physiological state and sample collection conditions. Furthermore, after acquiring a methylation alteration, the methylation pattern is generally conserved throughout disease progression. Compared to genetic mutations, DNAme patterns are easier to detect as they are binary signals (methylated or unmethylated) tend to occur in specific regions (CGIs) and can be easily amplified using PCR. In contrast, genetic alterations may vary considerably from patient to patient, even within the same cancer type, and can be spread over large sections of DNA, necessitating the need for more complex analytic tools.

The advantages of using DNAme as an OC biomarker are evident. However, this is still a relatively new area of research. No single DNAme marker has been shown to accurately detect OC alone. Identifying the methylation status of multiple markers simultaneously, rather than individual genes, will provide more sensitive and specific assays. With the advent of genome-wide array-based approaches, identification of a panel of circulating methylated biomarkers specific to OC is most likely to lead to the successful development of diagnostic and disease monitoring biomarkers.

Improved understanding of the origin and pathogenesis of HGSC and the role of DNAme in early cancer development, coupled with the recent surge in research surrounding the clinical application of cfDNA, has paved the way for the discovery and development of potential new HGSC-specific blood-based biomarkers. ctDNA biomarkers could prove valuable in the early detection and diagnosis of HGSC (currently problematic areas) and for disease monitoring for relapse during and following treatment. The use of ctDNA biomarkers could also potentially improve the specificity of OC screening. Furthermore, biomarker detection in body fluids may detect disease earlier than can be identified using imaging, again highlighting the promise of ctDNA detection for disease monitoring and for routine clinical follow up. Developing HGSC-specific DNAme biomarkers and assessing new technologies to facilitate the reliable detection of these markers in plasma is therefore justified.

Liquid biopsy samples are increasingly being adopted for a wide variety of applications in oncology. However, the use of these promising biomarkers precedes a core understanding of the mechanisms and dynamics underlying analytes and the resolution of important technical issues. Additional pre-clinical studies addressing the biology of liquid biopsy analytes are required. Furthermore, the majority of liquid biopsy assays lack evidence of clinical validity and utility. Implementation of novel multiparameter strategies to combine information from multiple sources will play a significant role in establishing liquid biopsies in the clinic.

The initial discovery phase of the candidate DNAme biomarkers identification was implemented using matched tissue samples from each stage of the carcinogenic pathway of HGSC: NFT, STIC and HGSC. Although the sample size was small, it provided a highly unique sample set from which to identify potential early detection HGSC-specific DNAme biomarkers. The identification of DNAme markers showing hypermethylation in STIC lesions was also an essential component in choosing potential HGSC-specific early detection biomarkers.

The present invention served to develop assays for the most promising HGSC-specific DNAme markers and evaluate their potential as blood-based biomarkers. Seven candidate DNAme markers were developed and extensively optimised by pyrosequencing analysis. The diagnostic accuracy of two DNAme markers (cg08202949 and cg09101017) surpassed that of the gold standard, CA125, in tissue samples. qMSP assays were developed and evaluated for 4 DNAme markers, two of which (cg08202949 and cg09101017) again showed improved accuracy in detecting HGSC compared to CA125. The bisulphite conversion step required for qMSP analysis posed a number of challenges, namely in degrading DNA quality and yield, and was therefore abandoned in favour of a technique that would avoid the need for bisulphite conversion, MSRE qPCR. Prohibitively low cfDNA yields obtained from the patient plasma samples lead to the development of a targeted MSRE pre-amplification strategy, with subsequent proof-of-concept validation of one marker, cg08202494, in a small plasma cohort. The DNAme marker distinguished the HGSC group from the NFT group using cfDNA extracted from plasma samples. The marker outperformed the current gold standard, CA125, in terms of specificity (cg08202494, specificity 86.67%; CA125, specificity 72.22%). However, the overall diagnostic accuracy of CA125 was higher (CA125 AUC 0.912; cg08202494. AUC 0.8646). As eluded to previously, it is unlikely that any single DNAme marker will possess the desired sensitivity and specificity required to accurately diagnose such as heterogeneous disease as HGSC, but a combination of such markers could have real potential as a diagnostic and/or disease monitoring tool, either complementing or superseding CA125.

Even under optimal conditions, it is unlikely that a single marker would fulfil the high specificity required for population-wide, early detection of OC. Herein, seven markers were observed to show statistically significant hypermethylation in HGSC tissue samples compared to NFT using pyrosequencing analysis. Due to constraints on resources and time, only one marker, cg08202494, was taken through the entire development process for evaluation in plasma samples. The remaining six markers warrant further investigation given that the combination of all seven markers achieved an AUC of 0.9946 using Pyrosequencing analysis. Importantly, all these markers showed statistically significant hypermethylation in stage I disease, therefore, may have clinical utility in detecting early stage disease. An adequately powered, prospective clinical study is required to evaluate the potential diagnostic and disease monitoring roles of these markers in HGSC.

A logistic regression classifier to predict HGSC was also developed. The classifier performed well in the Test dataset, correctly labelling all but one sample. Combining DNAme markers and diagnostic methods adds significant complexity to test development. The algorithm by which the measurements are combined to yield a diagnostic result critically influences the sensitivity and specificity of the test.

The present invention provides the development of HGSC-specific blood-based biomarkers, having successfully developed a MSRE qPCR assay for the DNAme marker cg08202494. This assay detected statistically significant hypermethylation in HGSC plasma samples compared to normal controls.

1. A method of diagnosing ovarian cancer in a patient, the method comprising the steps of:
(a) providing a biological sample from the patient;
(b) measuring the nucleic acid methylation levels of one or more biomarkers in the sample; and
(c) diagnosing ovarian cancer in the patient based on the nucleic acid methylation levels;
wherein the or each biomarker is a gene selected from KRT87P; OSR2; LINC01197; ZNF469; MAP3K8; LINC01798; PHACTR3; TNS3; TFAP2A/LINC00518; PRRX1; NR5A1; LHX9; RBPMS; TACC1; DNHD1; TGFB1 I1; CACNA1A; ZNF154; KRT87P; PHOX2B; SHANK1; GRM1; HOXA9; TCERG1L; SCGB1B2P/LOC643719; NEFM; BARHL2; ZIC1; CLIC6; HCG15; PCDHGA4; LINC01159; PCDHGA4; CLIC6; ZNF729; PANTR1/LINC01158; DLX1/DLX-DT; and OTX1.

2. A method according to claim 1, wherein the ovarian cancer is selected from fallopian tube cancer, primary peritoneal cancer, epithelial ovarian cancer; high grade serous carcinoma; serous tubal intraepithelial carcinoma (STIC), and serous tubal intraepithelial lesion (STIL).

3. A method according to claim 1 or 2, wherein the or each biomarker is a gene having a NCBI Reference Sequence Version Number selected from NR_146088.1; NM_001286841.1; NR_034095.1; NM_001367624.2; NM_001320961.2; NR_110156.1; NM_080672.5; NM_022748.12; NM_001372066.1, NR_027793.1; NM_006902.5; NM_004959.5; NM_020204.3; NM_001008710.3; NM_001352789.2; NM_144666.3; NM_001042454.3; NM_001127222.2; NM_001085384.3 and NM_001320198.2; NM_003924; NM_016148; NM_001278064; NM_152739; NM_174937; NR_027620; NM_005382; NM_020063; NM_003412; NM_001317009; NR_145490; NM_018917; NR_110373; NM_018917; NM_001317009; NM_001242680; NR_037883; NM_178120; and NM_014562

4. A method according to claim 1 or 2, wherein the or each biomarker is a gene having a NCBI Reference Sequence Version Number selected from NR_146088.1; NM_001286841.1; NR_034095.1; NM_001367624.2; NM_001320961.2; NR_110156.1; NM_080672.5; NM_022748.12; NM_001372066.1, NR_027793.1; NM_006902.5; NM_004959.5; NM_020204.3; NM_001008710.3; NM_001352789.2; NM_144666.3; NM_001042454.3; NM_001127222.2; NM_173632.4 and NM_001320198.2.

5. A method according to any one of claims 1-4, wherein the measuring step (b) comprises measuring a methyl group of a cytosine/guanine dinucleotide of the or each biomarker.

6. A method according to any one of claims 1-5, wherein the measuring step (b) comprises measuring a methyl group of one or more cytosine/guanine dinucleotide having a CpG cluster ID (cg #) selected from cg08202494; cg01657761; cg03035213; cg03314029; cg04453471; cg07215504; cg11469908; cg15712559; cg16329896; cg05224741; cg09010107; cg04043571; cg08610862; cg13912311; cg23044884; cg14284618; cg15511120; cg23910243; cg22187630; cg01268824; cg07078225; cg07907386; cg08447324; cg08958294; cg26365299; cg03109827; cg23200020; cg23290344; cg24884703; cg12595013; cg11528328; cg02766845; cg07489502; cg07316846; cg17754510; cg18617005; cg19200589; cg03692651; cg08189989; cg10293403; cg20935165; and cg25622366.

7. A method according to any one of claims 1-5, wherein the measuring step (b) comprises measuring a methyl group of one or more cytosine/guanine dinucleotide having a CpG cluster ID (cg #) selected from cg08202494; cg01657761; cg03035213; cg03314029; cg04453471; cg07215504; cg11469908; cg15712559; cg16329896; cg05224741; cg09010107; cg04043571; cg08610862; cg13912311; cg23044884; cg14284618; cg15511120; cg23910243; cg22187630; cg01268824; and cg07078225.

8. A method according to any one of claims 1-5, wherein the measuring step (b) comprises measuring a methyl group of one or more cytosine/guanine dinucleotide having a CpG cluster ID (cg #) cg07078225.

9. A method according to any one of claims 1-8, wherein the measuring step (b) comprises measuring a methyl group of a cytosine nucleotide of a cytosine/guanine dinucleotide of the or each biomarker.

10. A method according to any one of claims 1-9, wherein the measuring step (b) comprises measuring a methyl group of a nucleotide located at one or more of: chr8:99,961,381-99,961,763 (encompassing cg08202494); chr15:95,836,182-95,836,449 (encompassing cg01657761); chr16:88,496,985-88,497,220 (encompassing cg03035213); chr10:30,726,381-30,726,735 (encompassing cg04453471); chr16:88,496,945-88,497,180 (encompassing cg07215504); chr2:66,918,184-66,918,501 (encompassing cg11469908); chr20:58,180,486-58,180,815 (encompassing cg15712559); chr7:47,515,002-47,515,209 (encompassing cg16329896); chr6:10,422,232-10,422,455 (encompassing cg05224741); chr1:170,638,675-170,639,000 (encompassing cg09010107); chr9:127,265,620-127,266,359 (encompassing cg04043571); chr1:197,888,169-197,888,821 (encompassing cg08610862); chr9:127,265,221-127,265,590 (encompassing cg13912311); chr8:30,244,794-30,245,560 (encompassing cg23044884); chr8:38,627,724-38,628,106 (encompassing cg14284618); chr11:6,597,801-6,598,507 (encompassing cg15511120); chr16:31,484,429-31,484,901 (encompassing cg23910243); chr19:13,616,758-13,617,066 (encompassing cg22187630); chr19:58,220,672-58,220,980 (encompassing cg01268824); chr12:52,652,239-52,652,588 (encompassing cg07078225); chr4:41,749,299-41,749,620 (encompassing cg07907386); chr19:51,170,701-51,170,975 (encompassing cg08447324); chr6:146,350,003-146,350,304 (encompassing cg08958294); chr7:27,205,236-27,205,573 (encompassing cg26365299); chr10:133,110,251-133,110,489 (encompassing cg03109827); chr19:35,068,434-35,068,718 (encompassing cg23200020); chr8:24,771,352-24,771,634 (encompassing cg23290344); chr1:91,185,304-91,185,587 (encompassing cg24884703); chr3:14,712,800-14,712,337 (encompassing cg12595013); chr21:36,041,529-36,041,864 (encompassing cg11528328); chr6:28,956,451-28,956,779 (encompassing cg02766845); chr5:140,811,560-140,811,772 (encompassing cg07489502); chr2:105,484,613-105,484,838 (encompassing cg07316846); chr6:10,391,302-10,391,566 (encompassing cg17754510); chr5:140,787,378-140,787,678 (encompassing cg18617005); chr21:36,041,458-36,041,748 (encompassing cg19200589); chr19:22,444,471-22,444,757 (encompassing cg03692651); chr2:105,459,051-105,459,325 (encompassing cg08189989); chr6:10,421,540-10,421,849 (encompassing cg10293403); chr2:172,972,746-172,972,978 (encompassing cg20935165); and chr2:63,281,025-63,281,296 (encompassing cg25622366).

11. A method according to any one of claims 1-8, wherein the measuring step (b) comprises measuring a methyl group of a nucleotide located at one or more of: chr8:99,961,381-99,961,763 (encompassing cg08202494); chr15:95,836,182-95,836,449 (encompassing cg01657761); chr16:88,496,985-88,497,220 (encompassing cg03035213); chr10:30,726,381-30,726,735 (encompassing cg04453471); chr16:88,496,945-88,497,180 (encompassing cg07215504); chr2:66,918,184-66,918,501 (encompassing cg11469908); chr20:58,180,486-58,180,815 (encompassing cg15712559); chr7:47,515,002-47,515,209 (encompassing cg16329896); chr6:10,422,232-10,422,455 (encompassing cg05224741); chr1:170,638,675-170,639,000 (encompassing cg09010107); chr9:127,265,620-127,266,359 (encompassing cg04043571); chr1:197,888,169-197,888,821 (encompassing cg08610862); chr9:127,265,221-127,265,590 (encompassing cg13912311); chr8:30,244,794-30,245,560 (encompassing cg23044884); chr8:38,627,724-38,628,106 (encompassing cg14284618); chr11:6,597,801-6,598,507 (encompassing cg15511120); chr16:31,484,429-31,484,901 (encompassing cg23910243); chr19:13,616,758-13,617,066 (encompassing cg22187630); chr19:58,220,672-58,220,980 (encompassing cg01268824); chr12:52,652,239-52,652,588 (encompassing cg07078225).

12. A method according to any one of claims 1-8, wherein the predicting step comprises comparing the nucleic acid methylation level of the or each biomarker with the nucleic acid methylation level of a respective normal.

13. A method according to claim 10, wherein deviation of the nucleic acid methylation level of the or each biomarker from the nucleic acid methylation level of the respective normal is indicative of ovarian cancer.

14. A method according to claim 10 or 11, wherein a nucleic acid methylation level of the or each biomarker higher than the nucleic acid methylation level of the respective normal is indicative of ovarian cancer.

15. A method according to any one of claims 1-8, wherein the method comprises
(a) providing a first biological sample from the patient;
(b) measuring the nucleic acid methylation levels of one or more biomarkers in the first sample;
(c) providing a second or subsequent biological sample from the patient;
(d) measuring the nucleic acid methylation levels of one or more biomarkers in the second or subsequent sample; and
(e) comparing the nucleic acid methylation levels of the one or more biomarkers in the first sample with the nucleic acid methylation levels of one or more biomarkers in the second or subsequent sample.

16. A method according to claim 14, wherein deviation of the nucleic acid methylation level of the or each biomarker in the second or subsequent sample from the nucleic acid methylation level of the or each biomarker in the first sample is indicative of progression of ovarian cancer.

17. A method according to any one of claims 1-15, wherein the biological sample is selected from tissue, whole blood, serum, plasma, urine, interstitial fluid, peritoneal fluid, cervical sampling, tears, saliva, pleural fluid, intra-uterine lavage/fluid, vaginal lavage/fluid, and cerebrospinal fluid.


SEQUENCE LISTING (111 sequences)

SEQ ID NO: 1 - DNA, 27 bp, Unknown
  Synthetic Primer Sequence
attttattttaagtagggggttattgt

SEQ ID NO: 2 - DNA, 30 bp, Unknown
  synthetic primer sequence
taaactcaacttactcaaatctcccatctt

SEQ ID NO: 3 - DNA, 20 bp, Unknown
  synthetic primer sequence
tttttagttttgtataagga

SEQ ID NO: 4 - DNA, 25 bp, Unknown
  synthetic primer sequence
ttgtgttgttgatgttggaatgtag

SEQ ID NO: 5 - DNA, 24 bp, Unknown
  synthetic primer sequence
acaactaaaatttacataactcct

SEQ ID NO: 6 - DNA, 17 bp, Unknown
  synthetic primer sequence
tgatgttggaatgtagg

SEQ ID NO: 7 - DNA, 21 bp, Unknown
  synthetic primer sequence
ggggtaaggagtttattttga

SEQ ID NO: 8 - DNA, 21 bp, Unknown
  synthetic primer sequence
aaacctaccatcctcaactcc

SEQ ID NO: 9 - DNA, 25 bp, Unknown
  synthetic primer sequence
tgaagatagtgtagtagaagaatag

SEQ ID NO: 10 - DNA, 19 bp, Unknown
  synthetic primer sequence
gagggggaagtagttgaag

SEQ ID NO: 11 - DNA, 22 bp, Unknown
  synthetic primer sequence
acctcaaacccaaaactctacc

SEQ ID NO: 12 - DNA, 18 bp, Unknown
  synthetic primer sequence
agttgtttaggaaggatt

SEQ ID NO: 13 - DNA, 27 bp, Unknown
  synthetic primer sequence
agaggaatgttggattgtaggatgtta

SEQ ID NO: 14 - DNA, 29 bp, Unknown
  synthetic primer sequence
cccaaccatctacctctcaataaatttca

SEQ ID NO: 15 - DNA, 23 bp, Unknown
  synthetic primer sequence
catcattcacttacttaataatc

SEQ ID NO: 16 - DNA, 26 bp, Unknown
  synthetic primer sequence
tattgagaggtagatggttgggttat

SEQ ID NO: 17 - DNA, 22 bp, Unknown
  synthetic primer sequence
atactccactacaaaaccacct

SEQ ID NO: 18 - DNA, 16 bp, Unknown
  synthetic primer sequence
agatggttgggttatg

SEQ ID NO: 19 - DNA, 24 bp, Unknown
  synthetic primer sequence
tttaggaaggattttaggaagagg

SEQ ID NO: 20 - DNA, 30 bp, Unknown
  synthetic primer sequence
cttctactacaccatcttcaaaataaactc

SEQ ID NO: 21 - DNA, 15 bp, Unknown
  synthetic primer sequence
taggaagaggaaggt

SEQ ID NO: 22 - DNA, 25 bp, Unknown
  synthetic primer sequence
tagtgtttgtaaaagaaggggaaat

SEQ ID NO: 23 - DNA, 29 bp, Unknown
  synthetic primer sequence
ataaccttctaaacactttcttccatata

SEQ ID NO: 24 - DNA, 20 bp, Unknown
  synthetic primer sequence
ttgtaaaagaaggggaaata

SEQ ID NO: 25 - DNA, 26 bp, Unknown
  synthetic primer sequence
gtttgggtatttgtttttggaaggta

SEQ ID NO: 26 - DNA, 21 bp, Unknown
  synthetic primer sequence
actctacccaacacacctatc

SEQ ID NO: 27 - DNA, 19 bp, Unknown
  synthetic primer sequence
atttgtttttggaaggtag

SEQ ID NO: 28 - DNA, 26 bp, Unknown
  synthetic primer sequence
gagttggattaggtattgttttgaat

SEQ ID NO: 29 - DNA, 26 bp, Unknown
  synthetic primer sequence
cctatttacaaaaaaaccactatcct

SEQ ID NO: 30 - DNA, 25 bp, Unknown
  synthetic primer sequence
tgttttgaatttttttatattagag

SEQ ID NO: 31 - DNA, 28 bp, Unknown
  synthetic primer sequence
agatttgagtttttttttggttttttaa

SEQ ID NO: 32 - DNA, 21 bp, Unknown
  synthetic primer sequence
actaaaaaacatcccccatcc

SEQ ID NO: 33 - DNA, 21 bp, Unknown
  synthetic primer sequence
ttgttgtatagtttagagttt

SEQ ID NO: 34 - DNA, 28 bp, Unknown
  synthetic primer sequence
aaattttttttggaggaagtattagggt

SEQ ID NO: 35 - DNA, 30 bp, Unknown
  synthetic primer sequence
aacacaaacacctacaaacctttacaacaa

SEQ ID NO: 36 - DNA, 19 bp, Unknown
  synthetic primer sequence
caaacctttacaacaatcc

SEQ ID NO: 37 - DNA, 27 bp, Unknown
  synthetic primer sequence
tgtatatagaaataattggaaataggg

SEQ ID NO: 38 - DNA, 24 bp, Unknown
  synthetic primer sequence
ccccaaccctcctaaatcccctaa

SEQ ID NO: 39 - DNA, 20 bp, Unknown
  synthetic primer sequence
gaaataattggaaatagggt

SEQ ID NO: 40 - DNA, 20 bp, Unknown
  synthetic primer sequence
agggaaagaggggattgtat

SEQ ID NO: 41 - DNA, 25 bp, Unknown
  synthetic primer sequence
acccctactattccctaattttcaa

SEQ ID NO: 42 - DNA, 18 bp, Unknown
  synthetic primer sequence
agaggggattgtatatag

SEQ ID NO: 43 - DNA, 21 bp, Unknown
  synthetic primer sequence
tttttaggttgtggggggtta

SEQ ID NO: 44 - DNA, 28 bp, Unknown
  synthetic primer sequence
atcccttctaccccttccaaaaatacct

SEQ ID NO: 45 - DNA, 15 bp, Unknown
  synthetic primer sequence
gttgtggggggttag

SEQ ID NO: 46 - DNA, 29 bp, Unknown
  synthetic primer sequence
aaattttggaggtggattttagtaaatag

SEQ ID NO: 47 - DNA, 29 bp, Unknown
  synthetic primer sequence
aactcatttttccatatcaattcaataac

SEQ ID NO: 48 - DNA, 25 bp, Unknown
  synthetic primer sequence
attcaataactaccaaaaattacaa

SEQ ID NO: 49 - DNA, 29 bp, Unknown
  synthetic primer sequence
gtatgtagatttttttttaggggttgtag

SEQ ID NO: 50 - DNA, 30 bp, Unknown
  synthetic primer sequence
aaacataattcaactcccaaaaaaatttcc

SEQ ID NO: 51 - DNA, 25 bp, Unknown
  synthetic primer sequence
agattttttgttttttgaataattt

SEQ ID NO: 52 - DNA, 28 bp, Unknown
  synthetic primer sequence
gataagaaagggaagaatatgatgtaat

SEQ ID NO: 53 - DNA, 30 bp, Unknown
  synthetic primer sequence
ccaataaaaaaaaaacaaaaacctctttac

SEQ ID NO: 54 - DNA, 16 bp, Unknown
  synthetic primer sequence
atttgatttggggttg

SEQ ID NO: 55 - DNA, 18 bp, Unknown
  synthetic primer sequence
aggaggttgggtttgtgt

SEQ ID NO: 56 - DNA, 22 bp, Unknown
  synthetic primer sequence
aaccaaaaccaaacaaaacact

SEQ ID NO: 57 - DNA, 25 bp, Unknown
  synthetic primer sequence
ggttataaatattttttgttatttt

SEQ ID NO: 58 - DNA, 20 bp, Unknown
  synthetic primer sequence
atggggttgtagagtgttat

SEQ ID NO: 59 - DNA, 28 bp, Unknown
  synthetic primer sequence
aaacccaaaaaatatacaaacaatcaat

SEQ ID NO: 60 - DNA, 18 bp, Unknown
  synthetic primer sequence
gttgtagagtgttatggt

SEQ ID NO: 61 - DNA, 19 bp, Unknown
  synthetic primer sequence
cccgttttgactttgccaa

SEQ ID NO: 62 - DNA, 20 bp, Unknown
  synthetic primer sequence
ctcgagagggctttctgtcc

SEQ ID NO: 63 - DNA, 20 bp, Unknown
  synthetic primer sequence
tcaatttactgaggcccgag

SEQ ID NO: 64 - DNA, 20 bp, Unknown
  synthetic primer sequence
tccatccatcagcctaccac

SEQ ID NO: 65 - DNA, 21 bp, Unknown
  synthetic primer sequence
caggcacgtacagacctttgv

SEQ ID NO: 66 - DNA, 19 bp, Unknown
  synthetic primer sequence
cggtacctgagggccctag

SEQ ID NO: 67 - DNA, 21 bp, Unknown
  synthetic primer sequence
aggcctggactctcaactgtg

SEQ ID NO: 68 - DNA, 24 bp, Unknown
  synthetic primer sequence
actgcagaaatcagaccaaaagag

SEQ ID NO: 69 - DNA, 18 bp, Unknown
  synthetic primer sequence
tagaaccaccccagagag

SEQ ID NO: 70 - DNA, 20 bp, Unknown
  synthetic primer sequence
aagggaagggcgtctaggat

SEQ ID NO: 71 - DNA, 20 bp, Unknown
  synthetic primer sequence
aacttcaggcagctgttcag

SEQ ID NO: 72 - DNA, 18 bp, Unknown
  synthetic primer sequence
actcaagggcaagcggaa

SEQ ID NO: 73 - DNA, 19 bp, Unknown
  synthetic primer sequence
cgaggaggccaggtagaga

SEQ ID NO: 74 - DNA, 21 bp, Unknown
  synthetic primer sequence
agtcttggatagggaacaggt

SEQ ID NO: 75 - DNA, 21 bp, Unknown
  synthetic primer sequence
ctctgggattgttctctggga

SEQ ID NO: 76 - DNA, 19 bp, Unknown
  synthetic primer sequence
atggggttctgaaggctcc

SEQ ID NO: 77 - DNA, 19 bp, Unknown
  synthetic primer sequence
cactgctgcactgttcttg

SEQ ID NO: 78 - DNA, 19 bp, Unknown
  synthetic primer sequence
cctccagggcttgcttact

SEQ ID NO: 79 - DNA, 18 bp, Unknown
  synthetic primer sequence
gggcagccaggagaagtc

SEQ ID NO: 80 - DNA, 21 bp, Unknown
  synthetic primer sequence
ggaccaggaaacgacttaagg

SEQ ID NO: 81 - DNA, 20 bp, Unknown
  synthetic primer sequence
acatcctgatatcggctgca

SEQ ID NO: 82 - DNA, 19 bp, Unknown
  synthetic primer sequence
cgtccacatttccctccct

SEQ ID NO: 83 - DNA, 20 bp, Unknown
  synthetic primer sequence
ttgatttggactgtctggcg

SEQ ID NO: 84 - DNA, 20 bp, Unknown
  synthetic primer sequence
tcggagaccgaattcaaaat

SEQ ID NO: 85 - DNA, 22 bp, Unknown
  synthetic primer sequence
gctggttttcttctttctgttg

SEQ ID NO: 86 - DNA, 18 bp, Unknown
  synthetic primer sequence
aagcggagtttgccctgc

SEQ ID NO: 87 - DNA, 19 bp, Unknown
  synthetic primer sequence
actccttccttgacgctgc

SEQ ID NO: 88 - DNA, 20 bp, Unknown
  synthetic primer sequence
atcactccgcacgctattaa

SEQ ID NO: 89 - DNA, 21 bp, Unknown
  synthetic primer sequence
tcttttaatcgtcgttggcca

SEQ ID NO: 90 - DNA, 18 bp, Unknown
  synthetic primer sequence
ccagtggctttcgtgggc

SEQ ID NO: 91 - DNA, 20 bp, Unknown
  synthetic primer sequence
cccagctcattcacttgtca

SEQ ID NO: 92 - DNA, 21 bp, Unknown
  synthetic primer sequence
gtgtagatgtcggggtagtga

SEQ ID NO: 93 - DNA, 23 bp, Unknown
  synthetic primer sequence
tcctctgtcatactctagttcct

SEQ ID NO: 94 - DNA, 19 bp, Unknown
  synthetic primer sequence
catcctgtagcgcctcctg

SEQ ID NO: 95 - DNA, 20 bp, Unknown
  synthetic primer sequence
ccgtttcccttgcagtctag

SEQ ID NO: 96 - DNA, 20 bp, Unknown
  synthetic primer sequence
cgtctccaccctcagtactt

SEQ ID NO: 97 - DNA, 19 bp, Unknown
  synthetic primer sequence
agctcaccctgtccattcc

SEQ ID NO: 98 - DNA, 20 bp, Unknown
  synthetic primer sequence
taagcagcgtcaaggaagga

SEQ ID NO: 99 - DNA, 18 bp, Unknown
  synthetic primer sequence
ctctccgggtctctcagc

SEQ ID NO: 100 - DNA, 18 bp, Unknown
  synthetic primer sequence
gcagccttcaactccacg

SEQ ID NO: 101 - DNA, 18 bp, Unknown
  synthetic primer sequence
ccgatgcagcaaagaggc

SEQ ID NO: 102 - DNA, 18 bp, Unknown
  synthetic primer sequence
gcagcagagtccgacctc

SEQ ID NO: 103 - DNA, 20 bp, Unknown
  synthetic primer sequence
tgccccttccaatctgttcc

SEQ ID NO: 104 - DNA, 19 bp, Unknown
  synthetic primer sequence
cggtaggtatgcgggtcac

SEQ ID NO: 105 - DNA, 18 bp, Unknown
  synthetic primer sequence
ccctaccctccctgtcca

SEQ ID NO: 106 - DNA, 18 bp, Unknown
  synthetic primer sequence
tccccgtccagtggcttc

SEQ ID NO: 107 - DNA, 20 bp, Unknown
  synthetic primer sequence
tgaagtcaaggctgctctcg

SEQ ID NO: 108 - DNA, 18 bp, Unknown
  synthetic primer sequence
ggagcggggttggatcaa

SEQ ID NO: 109 - DNA, 20 bp, Unknown
  synthetic primer sequence
tccacaagtctaccgtctgt

SEQ ID NO: 110 - DNA, 20 bp, Unknown
  synthetic primer sequence
tcggctttcttttgcgaagg

SEQ ID NO: 111 - DNA, 18 bp, Unknown
  synthetic primer sequence
gaacgcgtgaaggtggtg